(19) United States
(12) Patent Application Publication
(10) Pub. No.: US 2024/0335487 A1
(43) Pub. Date: Oct. 10, 2024

(54) Multi-strain probiotic formulation for ameliorating or preventing symptoms of treatment resistant depression

(71) Applicant: Unique Biotech Limited, Hyderabad (IN)

(72) Inventors: Ratna Sudha Madempudi, Hyderabad (IN); Manoj Pandurang Dandekar, Hyderabad (IN); Manisurya Kumar Palepu, Hyderabad (IN); Srilakshmi Satya Satti, Hyderabad (IN)

(21) Appl. No.: 18/609,015

(22) Filed: Mar. 19, 2024

(30) Foreign Application Priority Data
Jun. 23, 2022 (IN) 202341018673

(51) Int. Cl. A61K 35/747; A61K 31/198; A61K 35/742; A61K 35/745; A61P 25/24

(52) U.S. Cl. CPC A61K 35/747; A61K 31/198; A61K 35/742; A61K 35/745; A61P 25/24

(57) ABSTRACT

Multi-strain probiotic formulation for ameliorating and preventing symptoms of treatment resistant depression

The current invention more specifically relates to methods and compositions comprising a multi-strain probiotic formulation that can be used for alleviating symptoms of treatment-resistant depression in humans.

The multi-strain probiotic formulation disclosed herein for use as treatment for treatment-resistant depression symptoms comprises Bacillus coagulans, Lactobacillus plantarum, Lactobacillus rhamnosus, Bifidobacterium lactis, Bifidobacterium breve, and Bifidobacterium infantis. More specifically, the multi-strain probiotic formulation comprises Bacillus coagulans Unique IS-2, Lactobacillus plantarum UBLP-40, Lactobacillus rhamnosus UBLR-58, Bifidobacterium lactis UBBLa-70, Bifidobacterium breve UBBr-01, and Bifidobacterium infantis UBBI-01.

### REFERENCE TO SEQUENCE LISTING

The substitute sequence listing is submitted as an ASCII formatted text filed via EFS-Web, with a file name of “Substitute Sequence Listing. TXT”, a creation date of Jul. 1, 2024, and a size of 14 kilobytes. The substitute sequence Listing filed via EFS-Web is a part of the specification and is incorporated in its entirety by reference herein

### FIELD OF INVENTION

The current invention relates to the field of probiotic formulation for alleviating symptoms of treatment-resistant depression (TRD). The current invention more specifically relates to methods of ameliorating, and/or preventing treatment-resistant depression symptoms by using a multi-strain probiotic formulation.

### BACKGROUND

According to the World Health Organization, more than 280 million people of all ages suffer from depression currently. Less than half of them receive appropriate treatment for their disorder, and, in low- and middle-income countries, 75% of people receive no treatment at all. The prevalence of depression varies from 0.4% to 15.7% in different countries of the world. Moreover, the efficacy of currently known antidepressant drugs still remains low, at around 70% (compared with a no-treatment recovery rate of around 50%). Also, the onset of effect of the medication is also prolonged, on average being around 4-6 weeks. With very few exceptions, drugs newly introduced into clinical practice are variants of existing drugs: progress towards the identification of novel targets to improve the clinical parameters associated with depression is very slow, and hence many depression cases are classified as treatment-resistant depression (TRD).

Most modern antidepressants do not differ in their mechanism of action from their predecessors, developed almost 70 years ago. Such drugs have several disadvantages and require long-term use. Moreover, approximately one-third of depressed patients are considered “treatment-resistant”.

Treatment-resistant depression refers to any form of depression or related disorder that in any one or more given individuals does not respond to at least one adequate trial with antidepressant therapy, or that responds to a lesser extent or displays a diminished or reduced response to said treatment when compared to depression or related disorder in a subject that displays no resistance to said treatment. Treatment-resistant depression (TRD) complicates the management of major depression (MD). The underlying biology of TRD involves interplay between genetic propensity and chronic and/or early life adversity.

In major depressive disorder (MDD), complete remission of symptoms is the optimal therapeutic goal. Remission occurs when the patient fully recovers psychosocial functioning with a minimal burden of residual effects. However, despite the rapid evolution of pharmacologic therapies over the past 50 years, research shows that only 60% to 70% of patients who are tolerant to antidepressants will respond to first-line monotherapy, and more than one-third of patients treated for depression will become treatment resistant.

In the past several years, the focus on treatment-resistant depression (TRD) has increased manyfold. Several studies have demonstrated that a significant proportion of patients treated for depression do not achieve full remission. In a meta-analysis of placebo-controlled, double-blind studies conducted by Fava and Davidson (Ref 1), data suggested that 29% to 46% of depressed patients treated with standard-dose antidepressants for at least 6 weeks failed to respond fully. Specifically, 12% to 15% of patients studied attained only a partial response, whereas 19% to 34% of this population was nonresponsive. In another meta-analysis, Golden et al. (Ref 2) reviewed 25 double-blind trials involving 4016 patients and found that more than 50% of patients treated with a single antidepressant failed to reach full remission. Even among patients considered to be full responders to a clinical trial of a single antidepressant, patients still experience a significant burden of residual symptoms such as insomnia and fatigue, which is also associated with an increased risk of relapse in 76% of patients studied.

Thus, there are unmet needs for newer therapeutic approaches which have fewer side effects and increased efficacy.

Many of the known neuropathologies of depression, such as monoamine neurotransmitters depletion, hyperactivation of the hypothalamic-pituitary-adrenal (HPA) axis, and immune imbalances, are interconnected with changes in gut microbiome. The crosstalk between the intestine and the brain referred to as the microbiome gut-brain axis, has also been increasingly reported in CNS disorders. Recent findings have reported an incidence of gut dysbiosis (i.e., imbalance in the microbial community) in depression, which may impact the integrity of the intestinal wall, increase the colonization of pathogenic bacteria, and also modulate the production of neurotransmitters, proinflammatory cytokines, neurotrophic factors, hormones, neuropeptides and tryptophan metabolism.

Several studies have demonstrated the importance of the gut microbiome in the neuropathology and management of depression. An altered gut microbiome signature has also been noted in depressive patients (Ref 3, Zheng et al), indicating the imbalance between beneficial and harmful bacterial colonies in the intestine of the host. Zheng et al (Ref 3) describes the causative association between major depressive disorder (MDD) and gut microbiota. There is increasing amount of scientific evidence demonstrating that gut microbiota can greatly influence all aspects of physiology. Emerging evidence also suggests that gut microbiota can influence brain function and behaviour through the ‘microbiota-gut-brain axis’ Jiang et al (Ref 4). Thus, the remodelling of commensal gut microbiota may become one of the treatment strategies to relieve depressive symptoms more effectively.

The profile of the gut microbiome can be modified using the administration of prebiotics, probiotics, postbiotics, and fecal microbiota transplantation (FMT). Interestingly, transplantation of depressive patients' fecal microbiota into germ-free (GF) mice showed depressive-like behavior. Therefore, the introduction of FMT as a beneficial microbial gut community from a healthy donor to depressive patients is also being investigated by several laboratories. In fact, administration of FMT from a healthy donor was reported to improve depressive symptoms after four weeks of treatment in two patients with major depressive disorder. In addition, several preclinical and clinical studies have also shown mood-elevating effects of probiotics via the microbiome-gut-brain axis.

The current invention encompasses the therapeutic potential and use of a multi-strain probiotic formulation in the management of TRD.

The multi-strain probiotic formulation disclosed herein contains a consortium of probiotic microbes, belonging to Bacillus sp., Lactobacillus sp., and Bifidobacterium sp. Specifically, the consortium is of probiotic Bacillus coagulans, Lactobacillus rhamnosus, Bifidobacterium lactis, Lactobacillus plantarum, Bifidobacterium breve and Bifidobacterium infantis. All the microbes are non-pathogenic to humans and animals. The probiotic formulation also comprises a non-essential amino acid “Glutamine”.

Specifically, the consortium is of probiotic Bacillus coagulans Unique IS-2 (MTCC 5260), Lactobacillus rhamnosus UBLR-58 (MTCC 5402), Bifidobacterium lactis UBBLa-70 (MTCC 5400), Lactobacillus plantarum UBLP-40 (MTCC 5380), Bifidobacterium breve UBBr-01 (MTCC 25081) and Bifidobacterium infantis UBBI-01 (MTCC 25124) and Glutamine (Ref 5, Dandekar et al).

The current invention encompasses a method of ameliorating symptoms associated with treatment-resistant depression, by treating subjects with a multi-strain probiotic formulation and glutamine. The current invention encompasses the use of a probiotic formulation comprising six unique bacterial strains in managing symptoms of TRD.

### SUMMARY

One embodiment of the current invention is a method of treating, preventing or ameliorating at least one symptom of treatment-resistant depression (TRD) in a subject, the method comprising the step of administering to the subject a multistrain probiotic formulation comprising the bacterial strains Bacillus coagulans. Lactobacillus rhamnosus. Bifidobacterium lactis. Lactobacillus plantarum. Bifidobacterium breve and Bifidobacterium infantis and 100-500 mg of Glutamine.

In one embodiment, the method described above, wherein the Bacillus coagulans is the strain Bacillus coagulans Unique IS-2 (MTCC 5260), Lactobacillus rhamnosus is the strain Lactobacillus rhamnosus UBLR-58 (MTCC 5402), Bifidobacterium lactis is the strain Bifidobacterium lactis UBBLa-70 (MTCC 5400), Lactobacillus plantarum is the strain Lactobacillus plantarum UBLP-40 (MTCC 5380), Bifidobacterium breve is the strain Bifidobacterium breve UBBr-01 (MTCC 25081) and Bifidobacterium infantis is the strain Bifidobacterium infantis UBBI-01 (MTCC 25124) in the multistrain probiotic formulation.

In one embodiment, the multistrain probiotic formulation comprises the bacterial strains Bacillus coagulans Unique IS-2 (MTCC 5260), Lactobacillus rhamnosus UBLR-58 (MTCC 5402), Bifidobacterium lactis UBBLa-70 (MTCC 5400), Lactobacillus plantarum UBLP-40 (MTCC 5380), Bifidobacterium breve UBBr-01 (MTCC 25081) and Bifidobacterium infantis UBBI-01 (MTCC 25124), and Glutamine.

In one embodiment, the TRD is induced by chronic stress or early-life stress. In one embodiment, the administration of an effective dosage regimen of the multistrain probiotic formulation to the subject does not cause any metabolic changes or locomotor changes in the subject.

In one embodiment, the multistrain probiotic formulation comprises Bacillus coagulans: Lactobacillus rhamnosus: Bifidobacterium lactis: Lactobacillus plantarum: Bifidobacterium breve and Bifidobacterium infantis in the ratio 2:2:2:2:1:1. The method of claim 1, wherein the bacterial strains are present in the multistrain probiotic formulation at 106-1012 colony forming unit (cfu) per dose.

In one embodiment, 1 to 2 doses of the multistrain probiotic formulation are administered to the subject per day. In one embodiment, the multistrain probiotic formulation is administered to the subject for at least 6 weeks. In one embodiment, the multistrain probiotic formulation is administered to the subject for 6-8 weeks.

In one embodiment, the method disclosed herein comprises the step of administering an effective dosage regimen of the multistrain probiotic formulation to the subject, wherein the effective dosage regimen comprises administering 1 to 2 doses per day of the multistrain probiotic formulation, for 6-8 weeks.

In one embodiment, the multistrain probiotic formulation is administered to the patient as an adjunct to standard medical treatment (SMT) for depression. In one embodiment, the multistrain probiotic formulation is administered orally to the patient.

In one embodiment, the multistrain probiotic formulation is administered to the subject in the form of a food product, a dietary supplement, or a pharmaceutically acceptable composition.

In one embodiment, the multistrain probiotic formulation alleviates treatment-resistant depression symptoms by increasing gut eubiosis in the patient.

In one embodiment, the administration of an effective dosage regimen of the multistrain probiotic formulation to the subject leads to an increase in the number of goblet cells and improved crypt-to-villi ratio in the colon of the subject.

In one embodiment, administration of an effective dosage of the probiotic formulation leads to amelioration of one or more of symptoms associated with TRD such as irritability, mood swings, depressed mood, disturbed sleep, listlessness, cognitive changes, short term memory loss, anxiousness, restlessness, tension, and anhedonia.

In one embodiment, administration of an effective dosage of the multistrain probiotic formulation leads to a decrease of the depression symptom of anhedonia in subjects treated with the formulation.

In one embodiment, the administration of the effective dosage regimen of multistrain probiotic formulation alleviates TRD symptoms by modulating the levels of L-kynurenine, kynurenic acid, and 3-hydroxy anthranilic acid tryptophan metabolites in the subject.

In one embodiment, the administration of the effective dosage regimen of multistrain probiotic formulation modulates the levels of tryptophan/kynurenine metabolic pathway metabolites.

In one embodiment, the administration of the effective dosage regimen of multistrain probiotic formulation restores the levels of the 5-HT in the subject with lowered 5-HT levels than normal.

In one embodiment, the administration of the effective dosage regimen of multistrain probiotic formulation restores the elevated levels of L-tryptophan, L-kynurenine, kynurenic acid, and 3-hydroxyanthranilic acid in the circulatory system of the subject.

In one embodiment, the administration of the effective dosage regimen of multistrain probiotic formulation reverses the accumulation of neurotoxic metabolites associated with anxiety or depression in the subject. In one embodiment, the neurotoxic metabolite is 3-hydroxy anthranilic acid.

In one embodiment, the administration of the effective dosage regimen of multistrain probiotic formulation reverses the decrease in levels of short-chain fatty acids associated with TRD in the subject. In one embodiment, the short-chain fatty acids are acetate, propionate and/or butyrate.

In one embodiment, the administration of an effective dosage regimen of the multistrain probiotic formulation to the subject decreases levels of proinflammatory cytokines in the subject. In one embodiment, the administration of an effective dosage regimen of the multistrain probiotic formulation reverses the elevated mRNA expression levels of TNF-α, and C-reactive protein associated with TRD in the subject.

In one embodiment, administration of an effective dosage regimen of the multistrain probiotic formulation reverses the elevated mRNA expression levels of CRP, TNF-α, and dopamine levels in the hippocampus and/or frontal cortex of subjects.

In one embodiment, the administration of an effective dosage regimen of the multistrain probiotic formulation to the subject leads to increase in the number of goblet cells and improved crypt-to-villi ratio in the colon of the subject.

In one embodiment, administration of an effective dosage regimen of the multi-strain probiotic formulation leads to a restores the expression of tight junction (TJ) proteins in the subject.

In one embodiment, administration of an effective dosage regimen of the multistrain probiotic formulation reverses the decrease in BDNF levels associated with TRD in the subject.

In one embodiment, administration of an effective dosage regimen of the multistrain probiotic formulation reverses the decrease in Firmicutes to Bacteroidetes ratio associated with TRD in the subject.

### BRIEF DESCRIPTION OF FIGURES AND SEQUENCE LISTING

FIG. 1 is a Schematic representation of the experimental study design. Rats were first subjected to MS between PND2 (Post Natal Day 2) through PND14 (Post natal Day 14) and then exposed to CUMS at the age of 12-14 weeks. The multi-strain probiotic treatment was initiated when animals were 8 weeks old for the next 6 weeks. The neurobehavioral assessment was conducted after six weeks of probiotics treatment.

FIG. 2 shows the effect of multi-strain probiotic formulation administration on sucrose consumption in MS+CUMS-exposed male and female rats using the Sucrose Preference

Test (SPT). The percentage of sucrose preference was calculated. The data (each bar) is represented as mean ±S.E.M for six to eight rats.

FIG. 3 shows the effect of multi-strain probiotic formulation treatment in the FST (forced swimming test) in stress-exposed (MS+CUMS) male and female rats. The immobility was recorded during the test period of 5 min. The data (each bar) is represented as mean+S.E.M for six to eight rats.

FIG. 4 shows the effect of the multi-strain probiotic formulation treatment on expression of TNF-α in the frontal cortex (A) and hippocampus (B) in male and female rats. The data were analyzed by one-way ANOVA, followed by post-hoc Dunnett's multiple comparisons test. The sex-specific correlation was assessed using two-way ANOVA, followed by Bonferroni's multiple comparisons test.

FIG. 5 shows the effect of the multi-strain probiotic formulation on CRP expression in the frontal cortex (A) and hippocampus (B) in male and female rats.

FIG. 6 shows the effect of the multi-strain probiotic formulation treatment on the concentration of DA (A), NE (B), and 5-HT (C) in the frontal cortex of male and female rats.

FIG. 7 shows the effect of the multi-strain probiotic formulation treatment on the abundance of intestinal bacterial population in fecal samples of male and female rats.

FIG. 8 shows the photomicrographs showing H&E staining in the distal part of colon sections of the vehicle control (male rats) [A], vehicle+stress (MS+CUMS) in male rats [B], probiotics+stress (MS+CUMS) in male rats [C], vehicle control (female rats) [D], vehicle+stress (MS+CUMS) in female rats [E], and (multi-strain probiotic formulation)+stress (MS+CUMS) in female rats [F]. The villi-to-crypt ratio (G) and the goblet cell count (H) are presented in the bar graph as mean ±S.E.M.

### SEQUENCE LISTING

SEQ ID NOS: 1-10 are the primers for amplifying the different types of bacterial phyla, Firmicutes and Bacteroidetes, Actinobacteria and Proteobacteria. The SEQ ID NOS are also given in Table 9.

### DETAILED DESCRIPTION

Depression and anxiety are two of the most prevalent mental health disorders. The main clinical features of depression are loss of interest, often accompanied by guilt, hopelessness, loss of appetite and insomnia. The World Health Organization (WHO) estimated that the prevalence of depression in the global population was as high as 4.4% in 2015, and the prevalence of anxiety disorders was estimated to be 3.6%. The Covid pandemic has exacerbated the prevalence of depression and anxiety. Moreover, depression and anxiety frequently co-occur.

Treatment-resistant depression refers to any form of depression or related disorder that in any one or more given individuals does not respond to at least one adequate trial with antidepressant therapy, or that responds to a lesser extent or displays a diminished or reduced response to said treatment when compared to depression or related disorder in a subject that displays no resistance to said treatment. Treatment-resistant depression (TRD) complicates the management of major depression (MD). The underlying biology of TRD involves interplay between genetic propensity and chronic and/or early life adversity.

Many instances of depression manifest in physical symptoms, especially in primary care settings, occurrences manifest themselves in several ways and have different clinical courses.

Patients with depression have been reported to have higher levels of serum proinflammatory cytokines and acute-phase reactants. Moreover, increased inflammatory mediators may serve as predictive biomarkers for resistance to antidepressant treatment. Hence, treatment-resistant MDD patients with higher inflammatory mediators can actually benefit from anti-inflammatory medications.

Depression is an affective disorder with a heterogeneous group of symptoms. The exact pathophysiological mechanisms for effectiveness of different types of treatments are not understood completely so far. Some of the factors that are being researched are dysfunctions on the hypothalamic-pituitary axis, neurotransmitters, vagus nerve, short-chain fatty acid metabolites, tryptophan, inflammatory factors and the brain-gut-microbiota axis. Many studies have shown that not only do stress-related disorders bring changes in the composition of the intestinal microbiota through the hypothalamic-pituitary-adrenal axis but the intestine also has an effect on the central nervous system through vagal stimulation, intestinal permeability and the release of inflammatory and anti-inflammatory compounds and changes in circulating agents in the blood. Many studies have also focused on the leaky gut syndrome contributing to neuropathological disorders.

The gut-brain axis refers to the bidirectional signalling mechanisms between the gastrointestinal tract and the central nervous system (CNS). Through complex neuro humoral pathways, signals from the brain can alter the sensorimotor and secretory functions of the gut, and conversely, visceral afferent signals originating in the gastrointestinal tract can modulate brain function. The gut microbiota is the ecological community of symbiotic and pathogenic microorganisms present in the gut, some of which can be critically involved in gut-brain communication. Imbalances in the microbial composition of the gut are present in gastrointestinal disorders such as Irritable Bowel Syndrome (IBS) and coeliac disease, and metabolic disorders such as obesity and diabetes, as well as in mental illnesses such as eating disorders, autism spectrum disorder (ASD) and mood and anxiety disorders.

Microbiota-gut-brain (MGB) communication can theoretically occur through multiple systems comprising the gut-brain axis (including the autonomic nervous system and enteric nervous system), neuroendocrine systems and the immune system. Nevertheless, the specific mechanisms of this communication and its putative effects on human brain development, behaviour, cognition and mood are largely unknown.

The main role of the intestinal barrier is in the digestion and absorption of nutrients: but it also controls the transport of antigens from the intestinal lumen into the submucosa, and maintains the balance between tolerance and the immune response to antigens that causes inflammation. The integrity of the intestinal barrier can be affected by diet, dysbiosis of the intestinal microbiota or by other factors. These have the potential to cause immune activation by translocation of microbial antigens and metabolites. (Ref 6, Iordache et al).

Many of the current research efforts are focused on the identification of specific microbial signatures for many inflammatory and metabolic diseases. more particularly for those associated with obesity, type 2 diabetes, and cardiovascular diseases. The relative abundance of Firmicutes, Bacteroidetes, Actinobacteria, and Proteobacteria has been reported between depressive people and healthy controls. A higher abundance of Firmicutes and Actinobacteria, and a lower abundance of Bacteroidetes have been reported in depression patients (Ref 7, Liu et al). Moreover, there is an association between Irritable bowel syndrome (IBS) and the incidence of depression and anxiety (Ref 6 & 7, Iordache et al & Liu et al). IBS is the most prevalent functional gastrointestinal disorder, affecting 10% of the population. Comorbidity of IBS with psychological distress is common. A higher ratio of Firmicutes/Bacteroidetes, has also been reported, leading to lower diversity in gut microflora. IBS patients had a high abundance of Bacteroides in mucosal microbiota, which was reduced after probiotic treatment. Jiang et al (Ref 4) reported that there is a relative greater abundance of, Bacteroidetes, Actinobacteria, and Proteobacteria in depressive people compared to healthy controls. The higher abundance of Firmicutes and Actinobacteria, and lower abundance of Bacteroidetes have been reported in depression patients. Jiang et al. also reported a lower abundance of Firmicutes in people with depression (Ref 4, Jiang et al) Thus, patients with depression have also been found to have microbiota dysbiosis. Therefore, microbiota balance might play an important role in supporting emotional well-being.

### Definitions

As used herein the term “and/or” encompasses “both” and “either/or”.

As used herein the term “multistrain probiotic formulation refers to a probiotic formulation comprising the probiotic species: Bacillus coagulans. Lactobacillus rhamnosus. Bifidobacterium lactis. Lactobacillus plantarum. Bifidobacterium breve and Bifidobacterium infantis. The formulation may optionally comprise 100-500 mg of Glutamine.

More specifically, the multi-strain probiotic formulation refers to the multistrain probiotic formulation described above, but with specific strains of these bacterial species, that is bacterial strains Bacillus coagulans Unique IS-2 (MTCC 5260), Lactobacillus rhamnosus UBLR-58 (MTCC 5402), Bifidobacterium lactis UBBLa-70 (MTCC 5400), Lactobacillus plantarum UBLP-40 (MTCC 5380), Bifidobacterium breve UBBr-01 (MTCC 25081) and Bifidobacterium infantis UBBI-01 (MTCC 25124), and Glutamine.

As used herein, “treating and preventing depression, a depressive related disorder” includes, the amelioration or alleviation, at least in part, of one or more symptoms of the depression, depressive disorder. For example, symptoms of depression, and related disorders that may be inhibited, or alleviated include irritability, mood swings, depressed mood, disturbed sleep, listlessness, cognitive changes, short-term memory loss, anxiousness, restlessness, tension, anhedonia, poor self-esteem, suicidal thoughts or suicidal tendencies.

As used herein, the term “treating” includes any effect, e.g., lessening, reducing, modulating, ameliorating or eliminating, that results in the improvement of the condition, disease, disorder, and the like, or ameliorating a symptom thereof.

Further, “treating or preventing” includes preventing the development of depression, treatment-resistant depression, or a depressive-related disorder in a subject that may be predisposed to such a condition or may display one or more symptoms of such a condition but has not yet been diagnosed with the condition. “Treating or preventing” also include preventing the onset of a depressive episode in a subject, or preventing the appearance of symptoms.

The term “treatment-resistant depression” as used herein refers to any form of depression or related disorder that in any one or more given individuals has not responded to at least one adequate trial with antidepressant therapy, or that responds to a lesser extent or displays a diminished or reduced response to said treatment when compared to depression or related disorder in a subject that displays no resistance to said treatment. Thus, “treatment-resistant depression” may indicate complete or partial resistance to treatment.

Chronic stress and early life stress are known to be major causative factors for TRD.

Thus, as used in the context of the current specification, an individual suffering from depression or a related disorder that does not respond to a particular treatment may be referred to a ‘non-responder’ with respect to that treatment, and an individual suffering from depression or a related disorder that does not respond fully to a particular treatment (i.e. shows some level of resistance to treatment such that response is diminished when compared to the response of an individual suffering from depression or a related disorder that does not display resistance to said treatment) may be referred to a ‘suboptimal-responder’ with respect to that treatment (displays ‘suboptimal treatment response’). The depression suffered by the individual may be referred to as treatment-resistant depression with respect to that treatment.

As used herein, the terms “composition” and “formulation are used interchangeably.

As used herein, the term “patient” and “subject” refers to the person or individual suffering from, or showing the symptoms of depression, specifically treatment-resistant depression (TRD), or a patient who is predisposed to TRD.

As used herein “effective dosage regimen” refers to the specific way a therapeutic drug is to be taken, including formulation, route of administration, dose, dosing interval, and treatment duration, so as to have the desired effect.

In the current invention “effective dosage regimen” of the multistrain probiotic formulation has the desired effect of preventing, treating and/or ameliorating TRD symptoms.

As used herein, the term “dose” refers to the measured and specific amount of a therapeutic composition, administered at one time. The number, and frequency of doses given over a specified period of time or prescribed intervals makes up the dosage regimen.

The term “conventional pharmaceuticals or compounds” or “conventional depression treatment therapy” or “Standard Medical Treatment” as used herein in the context of “other conventional pharmaceuticals or compounds used to treat depression or anxiety/stress disorder/symptoms” and refers to those pharmaceuticals or compounds that persons of skill in the art (including but not limited to physicians) conventionally use to treat the “condition/disorder/symptom” or “behavioural abnormality” associated with depression or anxiety/stress disorder.

The term “Anhedonia” as used herein refers to the reduced ability to experience pleasure. It may be social or physical anhedonia. Anhedonia is considered as a core feature of major depressive disorder.

As used herein, the term “effective amount” refers to the amount of a compound sufficient to effect beneficial or desired results. An effective amount can be administered in one or more administrations, applications or dosages and is not intended to be limited to a particular formulation or administration route.

“Therapeutically effective amount” includes an amount of a compound of the invention that is effective when administered alone or in combination to treat or prevent the onset of the disease condition or disorder. For a combination of compounds, each with a therapeutic effect, “therapeutically effective amount” of the combination of compounds is an amount of the combination of compounds claimed that is effective to treat the disease condition or disorder. The combination of compounds can be additive and is preferably a synergistic combination.

Depression or a depressive disorder is typically diagnosed only when the symptoms reach a threshold and last at least two weeks. A number of methods, tools and techniques that are well known to those skilled in the art are used for diagnosing depression, and depressive-related disorders, which may be treatment resistant; and for assessing the status or severity of the depression before and after therapeutic intervention, and their symptoms over time, for monitoring the change in status or severity of such conditions or symptoms over a period of time, including in response to treatment or therapy.

Such methods and techniques for diagnosing, assessing and monitoring depression, and depressive-related disorders that may be treatment-resistant, may comprise clinician assessment, self-assessment or self-reporting questionnaires, and clinician-completed reports or questionnaires, in addition to biochemical measurements. A variety of clinical measures of symptoms and mood are well-known to those skilled in the art.

Any of such method(s) or technique(s) in diagnosing depression, or a depressive or related disorder and for assessing or monitoring of the symptoms may be used, for example, for an initial determination of the suitability of an individual to be treated in accordance with the present disclosure or a determination of the efficacy of a treatment in accordance with the present disclosure in an individual.

Exemplary self-assessment or self-report questionnaires include, but are not limited to the Depression and Anxiety Stress Scale (DASS), the Outcome Questionnaire-45 (OQ45), Quality of Life in Depression Scale (QLDS, including a Quality of Life (QoL) score), the Beck Depression Inventory (BDI), the Warwick-Edinburgh Mental Well-Being Scale (WBS), the Mini International Neuropsychiatric Interview (MINI), the Structured Clinical Interview for DSM Disorders (SCID) and the Patient Health Questionnaire (PHQ, such as PHQ-9 and PHQ-2). Exemplary clinician-completed reports or questionnaires include, but are not limited to, the Hamilton Depression Rating Scale (HAM-D) and the Raskin Depression Rating Scale. Biochemical measurements that may be employed include, but are not limited to, whole blood serotonin levels.

The application of chronic stress predominately alters the neural activity in the frontal cortex and the hippocampus. The levels of important neurotransmitters such as dopamine (DA), serotonin (5-HT), and noradrenaline (NA) are observed to be changed in the brain of individuals affected by MDD or anxiety. The levels of Brain-derived neurotrophic factor (BDNF) and proinflammatory cytokines are also altered. The expression of neurogenesis marker doublecortin (DCX), a precursor protein expressed in immature neurons is usually affected following stressful stimuli. The occurrence of gut dysbiosis has also been linked with the appearance of pathogenic bacteria and increased expression of proinflammatory cytokines. This physiological event promotes the tryptophan metabolism by activating indoleamine-2,3-dioxygenase to the kynurenic acid pathway Chen et al (Ref 8). have reported that depressive behaviors induced by cytokines are related to the activation of the kynurenine pathway (KP). Studies based on animal models of depressive symptoms have reported that indoleamine 2,3-dioxygenase (IDO) is likely to be a key metabolic enzyme in the kynurenine pathway. Moreover, increased levels of TNF-α and IL-6 activate the enzymes of the kynurenine pathway, which convert the tryptophane to kynurenine.

Some of the major neuroendocrine indicators of stress are:
i. Hypothalamic-pituitary-adrenal (HPA) axis: Increased Cortisol secretion, failure to suppress Cortisol in response to dexamethasone—the dexamethasone suppression test (DST) and increased corticotropin (ACTH)/cortisol response to the combined dexamethasone/corticotropin-releasing hormone (DEX/CRH) test have been consistently associated with severe depression.
ii. The hypothalamic-pituitary-thyroid (HPT) axis is often abnormal in depression. The main abnormality is a chronobiological dysfunction of this axis as reflected by the decreased mean and amplitude of the 24-hour thyroid-stimulating hormone (TSH) secretion.
iii. Noradrenergic (NA) system. One of the most consistently reported abnormal findings in depression is a blunted growth hormone (GH) response to acute administration of clonidine, a partial α2-adrenoreceptor agonist, in drug-free patients.
iv. Polysomnographic sleep parameters: Depression is often associated with difficulties in initiation and maintenance of sleep, as well as poor subjective quality of sleep.

Also, stress and anxiety can lead to a significant decrease in the villi/crypt ratio and the number of goblet cells. Thus, there is a lot of evidence supporting the theory that the microbiome gut-brain axis and compromised intestinal integrity plays a crucial role in the pathophysiology of depression.

The current invention encompasses the therapeutic potential and use of a multi-strain bacterial probiotic formulation in the management of TRD.

The current invention discloses the use of multi-strain probiotic formulation comprising a consortium of probiotic microbes, belonging to Bacillus sp., Lactobacillus sp. and Bifidobacterium sp. for alleviation, treatment and prevention of TRD symptoms. Specifically, the consortium is of probiotic Bacillus coagulans Unique IS-2 (MTCC 5260), Lactobacillus rhamnosus UBLR-58 (MTCC 5402), Bifidobacterium lactis UBBLa-70 (MTCC 5400), Lactobacillus plantarum UBLP-40 (MTCC 5380), Bifidobacterium breve UBBr-01 (MTCC 25081) and Bifidobacterium infantis UBBI-01 (MTCC 25124). The probiotic formulation also comprises a non-essential amino acid “Glutamine”.

All the strains in this probiotic formulation were isolated and deposited in MTCC (Microbial type culture collection) and ATCC by Unique Biotech.

Bifidobacterium is a genus of gram-positive, anaerobic, and nonmotile bacteria. Similar to Lactobacillus. Bifidobacterium species are abundant occupants of the gastrointestinal tract. Bacillus coagulans is a spore-forming, lactic acid-producing, and facultative non-pathogenic anaerobic bacterium, which can survive in the extreme microenvironment of the intestine and germinate in the small intestine.

Lactobacillus plantarum is a gram-positive, nonmotile, non-spore-forming bacterium. Lactobacillus plantarum is a widely distributed lactic acid bacterium. It represents part of the microbiota of many foods and feeds, including dairy, meat, fish, vegetable fermented products (e.g., must, sauerkraut, pickled vegetables, sourdoughs), and silage: it is also a natural inhabitant of the human and animal mucosa (oral cavity, gastrointestinal tract, vagina, etc.) (Ref 9, Sulthana et al).

Lactobacillus rhamnosus is a gram-positive lactic acid bacterium that is part of the normal gut microflora in humans. It is generally regarded as safe and has been used extensively in food products and health supplements. L. rhamnosus is a gram-positive short heterofermentative anaerobe. In vitro studies in mice have revealed that L. rhamnosus is safe for human consumption (LD50 is greater than 50 g/kg/day).

The probiotic formulation disclosed also contains a non-essential amino acid glutamine. Glutamine is given as a precursor to the microbes so that they can utilize the amino acid and produce a useful metabolite, such as gamma-aminobutyric acid (GABA). Glutamine is the precursor to the formation of GABA in the brain. At least one or all of the said microbes in the probiotic formulation disclosed herein is capable of metabolizing glutamine to produce GABA. The amount of non-essential amino acid is 100 mg to 500 mg, preferably 200 mg to 300 mg per single dose of the formulation.

Role of Inflammation: Growing evidence supports the role of increased inflammation in psychiatric disorders. C-reactive protein (CRP) has also been reported to be elevated in depression disorder patients. CRP has also been reported to have moderate-to-strong associations with some other psychiatric disorders. Caldirola et al (Ref 10) have shown that eCRP (elevated CRP) may be a transdiagnostic indicator of proinflammatory status in a considerable portion of patients with psychiatric disorders, including depression. Hence there is documented association of increased CRP levels with greater illness severity, psychotropic treatment resistance, and increased cardiovascular risk.

Many studies have shown that the intestine also has an effect on the central nervous system through vagal stimulation, intestinal permeability and the release of inflammatory and anti-inflammatory compounds and changes in circulating agents in the blood. Many studies have also focused on the leaky gut syndrome contributing to neuropathological disorders.

The main role of the intestinal barrier is in the digestion and absorption of nutrients: but it also controls the transport of antigens from the intestinal lumen into the submucosa and maintains the balance between tolerance and the immune response to antigens that causes inflammation. The integrity of the intestinal barrier can be affected by diet, dysbiosis of the intestinal microbiota or by other factors. These have the potential to cause immune activation by translocation of microbial antigens and metabolites. (Ref 6, Iordache et al). Also, stress and anxiety can lead to a significant decrease in the villi/crypt ratio and the number of goblet cells.

Modelling Depression:
There are several models of depression (learned helplessness, chronic unpredictable mild stress, chronic social defeat stress, prenatal stress, maternal deprivation, social isolation, immune stimulation, corticosterone supplementation, drug-induced withdrawal, pharmacological, and genetic models) and tests to evaluate depressive-like behavior (forced swim test, sucrose preference test, open field, social interaction, tail suspension test, light-dark box test, elevated plus maze test, and intracranial self-stimulation)
Rodent models are widely used to study the neurobiological and genetic basis of depression given the availability of well-validated behavioral, physiological, and neuroendocrine assays (Ref 11, Cryan et al). Rats and mice are particularly well suited to studies of the effects of early life experience because they can be reared in large numbers under controlled environmental conditions. In the rat model, there is a large literature describing effects of stressors applied during the early postnatal period on behavioral and endocrine phenotypes in adulthood (Ref 12, Markov).

Early maternal separation (MS) as a model of depression:
Maternal separation early in life of a neonate, or MS, for varying periods of duration procedure fulfils the criteria for a depression model of high construct value. This model has mechanistic validity: the separation alters the regulation of the HPA axis in both humans and rats, in which the adult depressive-like behavior induced by MS180 is associated with HPA hyperactivity.
The neglect or maltreatment of children in early developmental periods increases the risk of anxiety, depression and psychoses in adulthood, which are often resistant to treatment. (Ref 13, Vetulani).

Chronic unpredictable mild stress (CUMS) is considered to be one of the most extensively validated animal models of depression. In this model, rats or mice are exposed chronically to a constant input of unpredictable micro-stressors, resulting in the development of a range of behavioural changes, including decreased response to rewards, a behavioural correlate of the clinical core symptom of depression, anhedonia. Reward sensitivity is usually tracked by periodic tests in which the animal is given access to a highly preferred sweet solution, or to a choice between a sweet solution and plain water. Consumption of, or preference for, the sweet reward decreases over weeks of exposure but can be restored to normal levels by chronic treatment with antidepressant drugs (Ref 14, Willner).

As an environmental stress induced depression model, CUMS paradigm mimics many aspects of depression and it is especially well known for producing anhedonia-like behavior, which can be easily assessed by sucrose preference or sucrose consumption tests. Furthermore, CUMS-induced depressive like behaviors only respond to chronic but not acute antidepressant treatments, which makes it one of the more realistic depression models. Besides, the association between neuroinflammation, immune mechanisms and depression has been well studied in CUMS model. CUMS-induced inflammation and inflammasome activation have also been widely reported (Ref 15, Aricioglu et al). In general, a single repeated stimulation triggers an adaptation response under normal conditions. However, the chronic mild stress (CMS) model can overcome this adaptation and produce long-term and effective depression in an animal group. This method for long-term inescapable stimulation comprises a series of trials, such as electric shock, ice walking, fixation, day and night reversal, tail clipping, and water or food deprivation, that are randomly performed every day.

Adult female rats that are subjected to dual MS and CUMS stressors exhibit more critical and intensive depression-like and anxiety-like behaviors, and this process has been hypothesized to result from synaptic plasticity impairment. These very intense depression-like and anxiety-like behaviors, which were more severe than the rats which underwent only MS or only CUMS (Ref 16 & 17, Huang et al & Bian et al). Behavioral tests in animal models have been developed primarily and specifically to verify and support theory of cognition or emotion: they can also be used to verify a theory of a psychopathology, these can be induced by depressants, and can be used to test anti-depressant therapies (Ref 18, Belovicova et al).

In the current study, considering chronic stress as a major risk factor for depression and alteration of stress-sensitive brain circuitry, the animals were subjected to MS+CUMS stress, which is one of the most reliable and an excellent translational animal model to study the negative effects of early- and late-life stress on neurological disorders.

The forced swimming test (FST: sometimes called Porsolt swim test) is a behavioral paradigm that is widely used as a screening test for antidepressant activity in rodents. FST is a common test used for evaluation of the efficacy of anti-depressant drugs and the effects of various behavioral and neurobiological manipulations in basic and preclinical research. It is a high throughput, easy to set up, has inter-laboratory reliability and specificity. Moreover, it has been applied to other species, including mice, Mongolian gerbils and sand rats (a species of gerbil).

Moreover, the limitations usually associated with the FST do not devalue the usefulness of FST as a drug discovery and validation tool (Ref 19 & 20, Can et al & Slattery et al).

The open-field test (OFT) was developed for testing animal emotionality in 1932. It was traditionally done with presenting to the animal an open field and food pellet placed in the middle. Animals slowly approach food in circular motions. However the tendency of animals to explore a new environment is decreased when food was removed. Although OFT was developed by behavioral psychologists, it is nowadays widely used in many science disciplines, including neuroscience or psychopharmacology.

### Elevated Plus Maze

The elevated plus maze (EPM) is a widely used procedure to assess anxiety-like behavior. EPM consists of 4 arms in cross shape with central zone in the middle, placed above the ground, at a height that may vary, ranging from 40 cm to 1 m approximately. The EPM consists of two open arms and two closed arms Rodents tend to avoid open and strongly illuminated places, but at the same time they tend to explore new spaces. Thus the ratio of these opposing stimuli is evaluated (Ref 21, Teegarden). The frequency of entries into the open, closed arms, the central zone and the total time spent in these zones is recorded.

### Sucrose Preference Test

The sensitivity to reward can be assessed by a simple sucrose preference test in which animals have access to water without and with different concentrations of sucrose, and the preference rate is then analyzed. This test is often used to assess the level of anhedonia behavior (Ref 21, Teegarden). The reduced interest in the reward caused e.g. by chronic stress, is a manifestation of depressive behavior.

There are at least four key biological molecules/systems underlying the pathophysiology of MDD: central dopamine, stress responses by the hypothalamic-pituitary-adrenal axis and autonomic nervous system, inflammation, and brain-derived neurotrophic factor. Animal experiments in several depression models have clearly indicated that gut microbiota is closely related to these molecules/systems and administration of probiotics and prebiotics may have beneficial effects on them. Although the results of microbiota profile of MDD patients varied from a study to another, multiple studies reported that bacteria which produce short-chain fatty acids such as butyrate and those protective against metabolic diseases (e.g., Bacteroidetes) were reduced (Ref 22, Kunugi). Large-scale clinical trials have revealed that a substantial proportion of patients with major depressive disorder (MDD) fail to respond to, or achieve remission with, the current first-line antidepressants. Also, many patients with MDD show poor adherence to the current antidepressants due to their adverse effects, which requires new treatment strategies with less adversity.

### Standard Treatment of Depression:

Depression, both unipolar and bipolar, is a “phasic” disease. Stressful life events can trigger depressive episodes, but the influence of such episodes may decrease over the course of the illness (Ref 23, Duval et al). It therefore is crucial to adequately treat depression in the early stages of the illness, in order to prevent morphological and functional abnormalities. Many reports suggest that a severely depressed patient needs antidepressant drug therapy and that a non-severely depressed patient may benefit from other “nonbiological” approaches, yet little research has been done on the effectiveness of different treatments for depression. Moreover, the antidepressant drugs may lead to different therapeutic response and tolerance in different individuals. The treatment of depressive illness does not stop with treatment of acute episodes, and has to be continuous therapeutic intervention, the optimal duration of which is still not fully elucidated.

Depression is both clinically and biologically a heterogeneous entity. Some of the standard treatments for different kinds of depression are listed below:

1. Antidepressant Drugs:
Tricyclic and tetracyclic antidepressants (TCAs): Serotonin reuptake inhibitors (SRI) and Noradrenaline reuptake inhibitors (NRI) such as clomipramine and amitriptyline, imipramine, doxepine, amoxapine, desipramine, maprotiline
Selective serotonin reuptake Inhibitors (SSRIs) such as citalopram, escitalopram, fluoxetine, fluvoxamine, paroxetine, sertraline,
Selective serotonin and noradrenaline reuptake inhibitors (SNRIs) such as venlafaxine, milnacipran, duloxetine.
Noradrenaline α2 receptor antagonist and serotonin 5-HT2 receptor antagonists such as mianserin, mirtazapine
Serotonin (5-HT2) receptor antagonists such as trazodone, nefazodone
Noradrenaline reuptake inhibitor such as reboxetine
Dopamine reuptake inhibitors such as bupropion, methylphenydate
Monoamine oxidase inhibitors (MAOIs)
   irreversible and nonselective: iproniazid
   reversible and selective: MAOI-A (moclobemide, toloxatone) MAOI-B (selegiline)
Melatonergic (MT1/2) receptor agonist and selective 5-HT2 receptor antagonist agomelatine
Others: Tianeptine, herbal medicine: hypericum perforatum (St. John's wort), amino acid derivative S-adenosylmethionine (SAM-e), tryptophan and 5 hydroxytryptophan dietary supplements

2. Mood Stabilizers
Lithium salts.
Antiepileptics such as carbamazepine/oxcarbazepine, sodium valproate/divalproate/
valpromide, lamotrigine

3. “NONCHEMICAL” THERAPIES such as Light therapy, Electroconvulsive therapy, Magnetic stimulation, Vagus nerve stimulation, Deep brain stimulation

4. PSYCHOTHERAPY such as Cognitive therapy, interpersonal psychotherapy, Problem-solving therapy, Psychodynamic-interpersonal psychotherapy, Psychoanalytic oriented psychotherapy Role of microbiota:

Dysbiosis of the gut microbiome plays a casual role in the neuropathology of psychiatric disorders, including anxiety and depression. Several studies have corroborated the therapeutic utility of CNS active probiotics. Among the several probiotics investigated to treat anxiety and depression, the Lactobacillus and Bifidobacterium genera have been shown to be more effective. Single strain such as Bifidobacterium longum or Bacillus coagulans and a combination of Lactobacillus and Bifidobacterium species are clinically useful for relieving the depressive behavior. However, identifying optimal strain combinations and underlying mechanisms responsible for microbiota-gut-brain interactions is largely unclear. Thus, the characterization of new probiotic combinations with their mechanism of action is gaining significance.

The current invention discloses the effect of a multi-strain probiotic formulation which contains six different bacterial strains, Bacillus coagulans Unique IS-2. Lactobacillus plantarum UBLP-40. Lactobacillus rhamnosus UBLR-58. Bifidobacterium lactis UBBLa-70. Bifidobacterium breve UBBr-01, and Bifidobacterium infantis UBBI-01, along with L-glutamine on treatment resistant depression-like behavior employing maternal separation (MS) and chronic-unpredictable mild stress (CUMS) model in rats. In the current study, the probiotic was administered for 6 weeks via drinking water, and anxiety- and depressive-like behaviors were assessed using the forced swim test (FST), sucrose preference test (SPT), elevated plus maze (EPM), and open-field test (OFT). Noteworthy, administration of multi-strain bacteria was expected to produce synergistic effects, as each strain may contribute differently by producing more beneficial short-chain fatty acids (SCFAs) or reversing the effects of pathogenic gut microorganisms. The six strains also work synergistically for increased production of GABA from the glutamine.

GABA is the main inhibitory NT in the brain and is formed through glutamate decarboxylase (EC 4.1.1.15) conversion of glutamate to GABA or is produced by commensal microorganisms from gut microbiota. Thus, it is widely accepted that low levels of GABA are responsible for the hyperexcitability of neurons. The glutamine-glutamate cycle in the nervous system is well known. Astrocytes and neurons are connected by a flow of glutamine (produced directly from glutamate) from astrocytes to neurons. In the neurons glutamine is converted to transmitter glutamate and GABA. However, after their release as transmitters most of the glutamate and a considerable amount of GABA are returned to astrocytes. This is the glutamine-glutamate (GABA) cycle (Ref 24, Hertz). Changes in GABAergic neurotransmission is associated with numerous CNS disorders, such as behavioral disorders, pain, and sleep, stress and depression. Impaired GABA homeostasis has been linked to several neurological disorders (e.g., autism spectrum disorders, schizophrenia, epilepsy and neurodegenerative diseases).

Numerous studies have highlighted the importance of low-grade inflammation in the pathophysiology of depression, such as increased levels of proinflammatory cytokines in the hippocampus. Moreover, inflammatory conditions promote the metabolism of tryptophan along the kynurenine pathway at the expense of the 5-HT pathway. The metabolites of this pathway contribute to neuroprotective or neurodegenerative changes in the brain. Moreover, mice submitted to chronic stress have been shown to have altered gut microbiota composition, decreased Lactobacillus species, and increased kynurenine levels. Restoration of gut Lactobacillus levels was found to be sufficient in improving metabolic changes and behavioral abnormalities. Therefore, there is an interplay of chronic inflammation, the kynurenine pathway, and depression, the plasma concentrations of L-tryptophan, L-kynurenine, kynurenic acid, and 3-hydroxyanthranilic acid in TRD affected patients.

The alteration of the HPA axis and microbiome-gut-brain axis has been reported in both MS and CUMS paradigms of depression. In a rat maternal separation (MS) model of depression, altered colonic functions such as increased ion transport and macromolecular permeability, and increased adhesion/penetration of total bacteria were observed together with an elevated corticosterone level in the blood.

In rodents, the application of MS+CUMS induces behavioral and neurobiological changes resembling that of depressive patients. Early-life stress given in the form of MS develops neurobehavioral aberrations, including anxiety- and depression-like behaviors. Moreover, the MS model is sensitive to gut microbiome-based therapeutics. Altered gut microbiota phenotypes have also been reported in the CUMS model of depression. FMT from CUMS-exposed mice led to anxiety- and depression-like behaviors in the recipient mice via the gut microbiota-inflammation-brain axis. The gender of depressive patients also differentially influences the gut microbiota diversity. For instance, increased relative abundance of Actinobacteria in females and decreased Bacteroidetes in males have been reported.

### EMBODIMENTS

In one embodiment, the current invention encompasses the use of a multi-strain probiotic formulation to treat, ameliorate and prevent symptoms of treatment resistant depression.

In one embodiment, the multi-strain formulation comprises six strains of probiotic strains, and Glutamine.

One embodiment of the current invention is a method of treating, preventing or ameliorating at least one symptom of treatment-resistant depression (TRD) in a subject, the method comprising the step of administering to the subject a multistrain probiotic formulation comprising the bacterial strains Bacillus coagulans. Lactobacillus rhamnosus. Bifidobacterium lactis. Lactobacillus plantarum. Bifidobacterium breve and Bifidobacterium infantis and 100-500 mg of Glutamine.

In one embodiment, the TRD is induced by chronic stress or early-life stress. In one embodiment, the administration of an effective dosage regimen of the multistrain probiotic formulation to the subject does not cause any metabolic changes or locomotor changes in the subject.

In one embodiment, treatment with the probiotic formulation does not affect body weight of the subject.

In one embodiment, the method described above, wherein the Bacillus coagulans is the strain Bacillus coagulans Unique IS-2 (MTCC 5260), Lactobacillus rhamnosus is the strain Lactobacillus rhamnosus UBLR-58 (MTCC 5402), Bifidobacterium lactis is the strain Bifidobacterium lactis UBBLa-70 (MTCC 5400), Lactobacillus plantarum is the strain Lactobacillus plantarum UBLP-40 (MTCC 5380), Bifidobacterium breve is the strain Bifidobacterium breve UBBr-01 (MTCC 25081) and Bifidobacterium infantis is the strain Bifidobacterium infantis UBBI-01 (MTCC 25124) in the multistrain probiotic formulation.

In one embodiment, the multistrain probiotic formulation comprises the specific bacterial strains Bacillus coagulans Unique IS-2 (MTCC 5260), Lactobacillus rhamnosus UBLR-58 (MTCC 5402), Bifidobacterium lactis UBBLa-70 (MTCC 5400), Lactobacillus plantarum UBLP-40 (MTCC 5380), Bifidobacterium breve UBBr-01 (MTCC 25081) and Bifidobacterium infantis UBBI-01 (MTCC 25124), and Glutamine.

In one embodiment, the multistrain probiotic formulation comprises Bacillus coagulans: Lactobacillus rhamnosus: Bifidobacterium lactis: Lactobacillus plantarum: Bifidobacterium breve and Bifidobacterium infantis in the ratio 2:2:2:2:1:1. The method of claim 1, wherein the bacterial strains are present in the multistrain probiotic formulation at 106-1012 colony forming unit (cfu) per dose.

In one embodiment, 1 to 2 doses of the multistrain probiotic formulation are administered to the subject per day. In one embodiment, the multistrain probiotic formulation is administered to the subject for at least 6 weeks. In one embodiment, the multistrain probiotic formulation is administered to the subject for 6-8 weeks.

In one embodiment, the method disclosed herein comprises the step of administering an effective dosage regimen of the multistrain probiotic formulation to the subject, wherein the effective dosage regimen comprises administering 1 to 2 doses per day of the multistrain probiotic formulation, for 6-8 weeks.

In one embodiment, the multistrain probiotic formulation is administered to the patient as an adjunct to standard medical treatment (SMT) for depression. In one embodiment, the multistrain probiotic formulation is administered orally to the patient.

In one embodiment, the multistrain probiotic formulation is administered to the subject in the form of a food product, a dietary supplement, or a pharmaceutically acceptable composition.

In one embodiment, the multistrain probiotic formulation alleviates treatment-resistant depression symptoms by increasing gut eubiosis in the patient.

In one embodiment, the administration of an effective dosage regimen of the multistrain probiotic formulation to the subject leads to an increase in the number of goblet cells and improved crypt-to-villi ratio in the colon of the subject.

In one embodiment, administration of an effective dosage of the probiotic formulation leads to amelioration of one or more of symptoms associated with TRD such as irritability, mood swings, depressed mood, disturbed sleep, listlessness, cognitive changes, short term memory loss, anxiousness, restlessness, tension, and anhedonia.

In one embodiment, administration of an effective dosage of the multistrain probiotic formulation leads to a decrease of the depression symptom of anhedonia in subjects treated with the formulation.

In one embodiment, the administration of the effective dosage regimen of multistrain probiotic formulation alleviates TRD symptoms by modulating the levels of L-kynurenine, kynurenic acid, and 3-hydroxy anthranilic acid tryptophan metabolites in the subject.

In one embodiment, the administration of the effective dosage regimen of multistrain probiotic formulation modulates the levels of tryptophan/kynurenine metabolic pathway metabolites.

In one embodiment, the administration of the effective dosage regimen of multistrain probiotic formulation restores the levels of the 5-HT in the subject with lowered 5-HT levels than normal.

In one embodiment, the administration of the effective dosage regimen of multistrain probiotic formulation restores the elevated levels of L-tryptophan, L-kynurenine, kynurenic acid, and 3-hydroxyanthranilic acid in the circulatory system of the subject.

In one embodiment, the administration of the effective dosage regimen of multistrain probiotic formulation reverses the accumulation of neurotoxic metabolites associated with anxiety or depression in the subject. In one embodiment, the neurotoxic metabolite is 3-hydroxy anthranilic acid.

In one embodiment, the administration of the effective dosage regimen of multistrain probiotic formulation reverses the decrease in levels of short-chain fatty acids associated with anxiety or depression in the subject. In one embodiment, the short-chain fatty acids are acetate, propionate and/or butyrate.

In one embodiment, the administration of an effective dosage regimen of the multistrain probiotic formulation to the subject decreases levels of proinflammatory cytokines in the subject. In one embodiment, the administration of an effective dosage regimen of the multistrain probiotic formulation reverses the elevated mRNA expression levels of TNF-α, and C-reactive protein associated with anxiety and depression in the subject.

In one embodiment, administration of an effective dosage regimen of the multistrain probiotic formulation reverses the elevated mRNA expression levels of CRP, TNF-α, and dopamine levels in the hippocampus and/or frontal cortex of subjects.

In one embodiment, the administration of an effective dosage regimen of the multistrain probiotic formulation to the subject leads to increase in the number of goblet cells and improved crypt-to-villi ratio in the colon of the subject.

In one embodiment, administration of an effective dosage regimen of the multi-strain probiotic formulation leads to a restoration of levels of tight junction (TJ) proteins in the subject.

In one embodiment, administration of an effective dosage regimen of the multistrain probiotic formulation reverses the decrease in BDNF levels in the subject.

In one embodiment, administration of an effective dosage regimen of the multistrain probiotic formulation reverses the decrease in Firmicutes to Bacteroidetes ratio in subjects showing symptoms of TRD.

In one embodiment treatment with the formulation disclosed herein causes restoration of intestinal functions.

In one embodiment, the multi-strain probiotic formulation ameliorates TRD symptoms by reshaping the microbiome gut-brain activity in both sexes.

In one embodiment, the probiotic formulation comprising a multi-strain probiotic formulation, helps make TRD affected patients more susceptible to conventional treatments for depression.

The multi-strain probiotic formulation disclosed herein contains a consortium of probiotic microbes, belonging to Bacillus sp., Lactobacillus sp. and Bifidobacterium sp. Specifically, the consortium is of probiotic Bacillus coagulans Unique IS-2, Lactobacillus rhamnosus UBLR-58, Bifidobacterium lactis UBBLa-70, Lactobacillus plantarum UBLP-40, Bifidobacterium breve UBBr-01 and Bifidobacterium infantis UBBI-01. All the microbes are non-pathogenic to humans and animals. The probiotic formulation also comprises a non-essential amino acid Glutamine.

Specifically, the consortium is of probiotic Bacillus coagulans Unique IS-2 (MTCC 5260), Lactobacillus rhamnosus UBLR-58 (MTCC 5402), Bifidobacterium lactis UBBLa-70 (MTCC 5400), Lactobacillus plantarum UBLP-40 (MTCC 5380), Bifidobacterium breve UBBr-01 (MTCC 25081) and Bifidobacterium infantis UBBI-01 (MTCC 25124) and Glutamine.

In one embodiment, the probiotic formulation is administered in combination with other therapeutic or ameliorative agents for treating the symptoms of treatment resistant depression.

In one embodiment, probiotic formulation described herein and the at least one additional agent are administered as a single composition or formulation.

In one embodiment, the method described herein is employed as an adjunct to one or more other treatments or therapies for depression, anxiety or depressive or anxiety-related disorders.

In one embodiment, administration of the multi-strain probiotic formulation leads to decrease in any one or more of the symptoms associated with stress in a subject/patient diagnosed with treatment resistant depressive disorder.

In one embodiment, administration of effective dosage of the probiotic formulation comprising the multi-strain probiotic formulation disclosed herein leads to amelioration of one or more of somatic symptoms associated with TRD, such as gastrointestinal disorders (IBS), back pain, chest pain, shortness of breath, heart palpitations, problems with sleep or appetite, and fatigue.

In one embodiment, administration of effective dosage of the probiotic formulation leads to amelioration of one or more of symptoms associated with TRD, such as, irritability, mood swings, depressed mood, disturbed sleep, listlessness, cognitive changes, short term memory loss, anxiousness, restlessness, tension, and anhedonia.

In one embodiment, the multi-strain probiotic formulation treatment positively impacts the intestinal epithelium and integrity.

In one embodiment, treatment with multi-strain probiotic formulation also leads to normalization/reversal of the shift from the synthesis of 5-HT from tryptophan to the kynurenine pathway in patients suffering from TRD, which leads to the accumulation of neurotoxic metabolites like 3-hydroxy anthranilic acid in patients suffering from TRD.

In one embodiment, treatment with the multi-strain probiotic formulation leads to restoration of the intestinal permeability associated with healthy individuals. In one embodiment, the treatment leads to a restoration of levels of tight junction (TJ) proteins in people with TRD, and restoration of the levels of goblet cells and tight junction proteins which are reduced in stressed, depressed and anxious subjects. This impact in affected individuals is correlated with the reduction of goblet cells and TJ proteins such as occludin, ZO-1, and claudin-1 in the colonic mucosa. Gut histology altered by depression, which may be TRD, is restored well by the multi-strain probiotic formulation treatment.

Moreover, reshaping the intestinal microbial ecosystem by probiotic ingestion positively affects mood behaviour. Short chain fatty acids (SCFAs) such as acetate, butyrate, and propionate are the microbial metabolites by-products that can enter into the systemic circulation, cross the blood-brain barrier, and modulate the neural circuitry in the brain. The reduced levels of SCFA were found to be reduced in the macaques depression model and in depressive patients. Moreover, decreased levels of acetic acid and propionic acid are also reported in the chronic stress model of mice and depressive patients.

In some embodiments, the present disclosure encompasses a method for treating, preventing or ameliorating at least one symptom of depression or a depressive disorder, comprising administering to a subject an effective dose of the multi-strain probiotic formulation, by modulating (e.g., increasing) the amount of GABA produced in the subject's gut by administering the probiotic formulation.

The present disclosure also provides methods of treating a subject in need thereof comprising administering to a subject a therapeutic composition comprising GABA-producing bacteria. As set forth herein, the GABA-producing bacteria can produce GABA in the subject's gut. The GABA can diffuse into other systems of the subject's body (e.g., the circulatory and nervous systems). There, the endogenous GABA can act as a neurotransmitter. In some embodiments, increased levels of GABA (e.g., in the nervous system) can help to alleviate the symptoms of depressive disorder.

A composition in the form of a prebiotic, probiotic or a medical food, the composition comprising at least one purified bacterial population that produces GABA at a pH range of between 4.5 and 7.5.

In one embodiment, the probiotic formulation disclosed herein is used for treatment of TRD in both male and female patients. In one embodiment, it exhibits equal positive effects in both sexes.

Pharmaceutical preparations comprising the multistrain probiotic formulations disclosed herein may be in the form of solids such as tablets, granules, powders, capsules, and liquids such as solutions, emulsions, suspension and the like.

Pharmaceutically acceptable or appropriate carriers for use in the pharmaceutical preparations comprising the multistrain probiotic formulations disclosed herein can be, but are not limited to, organic or inorganic, solid or liquid excipients which is suitable for the selected mode of application such as oral application or injection, and administered in the form of a conventional pharmaceutical preparation. Examples of carriers or excipients in the preparation include, but are not limited to starch, lactose, glucose, sucrose, dextrin, cellulose, paraffin, fatty acid glyceride, water, alcohol, gum arabic and the like. If necessary, auxiliary, stabilizer, emulsifier, lubricant, binder, pH adjustor controller, isotonic agent and other conventional additives may be added.

The current invention also encompasses methods for making and using this formulation, and also encompasses compositions comprising this formulation and carriers, additives, synbiotics or any other components required for administration of this formulation.

### EXAMPLES

In this study, MS+CUMS-generated behavioral aberrations were significantly ameliorated by 6 weeks of treatment with a multi-strain mixture of probiotics+L-glutamine, as rats displayed a robust antidepressant- and antianhedonic like phenotypes in the FST and SPT assays. Moreover, multi-strain probiotic formulation-treated animals showed increased entries and spent more time in the open arms of EPM, which indicates the reduced anxiety-like behavior. Moreover, no notable changes in body weight and locomotor activity was observed. Hence, the effect of the multi-strain probiotic formulation on metabolic and locomotor activity was ruled out as an earlier study has indicated that ingestion of multi-strain probiotic formulation for 28 days showed stress relieving effects in students who were undertaking examination (Ref 25, Venkataraman et al).

### Example 1: Multistrain Probiotic Formulation Preparation and GABA Producing Activity

Testing of different ratios and combinations of the different strains was done to find out the optimal combination with the highest synergistic effect for producing GABA. The data and methodology are given below.

### Microbial Strains:

A deposit of the six microbial strains of the multistrain probiotic formulation has been made in a depository affording permanence of the deposit and ready accessibility thereto by the public if a patent is granted. The strains with their corresponding accession numbers are given below:
i Bacillus coagulans Unique IS-2: MTCC-5260
ii Lactobacillus rhamnosus UBLR-58: MTCC 5402
iii Bifidobacterium lactis UBBLa-70: MTCC 5400
iv Lactobacillus plantarum UBLP-40: MTCC 5380
v Bifidobacterium breve UBBr-01: MTCC 25081
vi Bifidobacterium infantis UBBI-01: MTCC 25124

The Microbial Type Culture Collection and Gene Bank (MTCC facility in IMTECH, Sector-39, Chandigarh, India-160036) (https://mtccindia.res.in/), is an affiliate member of the World Federation for Culture Collections (WFCC) and is registered with the World Data Centre for Microorganisms (WDCM).

The experiment given below was done to test various combinations of the six different probiotic strains.

### Methodology

Bacteria were inoculated in MRS broth containing 1% (w/v) mono sodium glutamate and incubated at 37° C. for 24 h. The culture was centrifuged at 10000×g for 10 min and 100 μL of supernatant was mixed with 400 μL of methanol and dried at 70° C. Approximately, 1 mL LaCl3 (Lanthanum chloride) was added in to the tube, mixed thoroughly by vortexing and centrifuged at 10000×g for 5 min. The supernatant (700 μL) was collected and mixed with 160 μL of 0.1 M KOH. The mixture was then centrifuged and 200 μL of supernatant was diluted with potassium pyrophosphate buffer. From this, 550 μL of sample was measured for GABA. Each sample was added with 200 μL 0.5 M K4P207, 150 μL 4 mM NADP, and 50 μL 2.0 units/mL Gabase (Sigma-Aldrich, USA). Initial absorbance was measured at 340 nm. Then 50 μL of 20 mM ketoglutaric acid was added and incubated at room temperature for 1 h. Final absorbance was measured at 340 nm to calculate the concentration.

### Glutaminase Assay

Bacteria were cultivated and supernatant was obtained as described above. In brief, 500 μL of 0.04 M of L-glutamine prepared in 0.1 M phosphate buffer (pH 7.0) was mixed with 500 μL of supernatant and incubated at 37° C. for 30 min. Enzyme reaction was stopped by adding 500 μL of 1.5 M tri-chloro acetic acid. Then equal volume of Nessler's reagent (Potassium tetraiodomercurate-Potassium hydroxide solution: Sigma-Aldrich, USA) was added and incubated for 15 min or until appearance of yellow colour. Samples were centrifuged and absorbance of supernatant was measured at 450 nm. Ammonium sulphate was used as the standard. One IU of glutaminase was defined as the amount of enzyme that liberates one micromole of ammonia under optimum conditions.

### Multistrain Probiotic Formulation GABA Producing Synergistic Activity:

It can be seen, as shown in Table 4, that the GABA producing activity of the four strains Bifidobacterium infantis UBBI-01, Bifidobacterium breve UBBr-01, B. coagulans Unique IS-2 and B. lactis UBBLa-70 (33.29, 25.85, 25.30 and 17.30 μM/ml respectively) is the highest amongst all the strains, and the other two strains Lactobacillus plantarum UBLP-40 and Lactobacillus rhamnosus UBLR-58a do not individually exhibit GABA producing activity. The combination of these four high GABA producing strains was tested (Table 4), and it was found, that unexpectedly, this combination of four high GABA producing strains does not show GABA producing activity equivalent to the combination of six strains (Table 1). The combination of these four strains which produce GABA individually at high levels, shows GABA producing activity of 33.18 μM/ml (Table 4), while when these four strains are combined together with the 2 other strains that individually do not show GABA producing activity, the resulting combination of the six strains shows GABA producing activity of 45.35 μM/ml (Table 1). Thus, it is clear that the two strains that do not individually show GABA producing activity, when present as a combination in the unique formulation disclosed in the current invention, increase the effect of the other four strains. Also, combinations of two and three strains shown in Tables 2 and 3, shows much lesser GABA producing activity of 29.73 and 18.78 μM/ml. Therefore, a synergistic effect is clearly evident in the unique combination of the six strains.

The ratio of the different strains was also finalised based on their GABA producing activity and subsequent experiments were done to validate the particular ratios. Bifidobacterium breve UBBR-01 and Bifidobacterium infantis UBB101 were found to produce the highest amounts of GABA and hence proportions of both these strains in the total strength of 10 billion colony forming units (cfu) was taken to be 1 billion cfu each, or in a ratio of 1:1. The remaining four strains were taken at 2 billion cfu each in the total strength of 10 billion cfu/capsule with a ratio of 2:2:2:2. Therefore, the ratio of the strains L. plantarum UBLP-40 (MTCC 5380): L. rhamnosus UBLR-58 (MTCC 5402): B. coagulans Unique IS-2 (MTCC 5260): B. lactis UBBLa-70 (MTCC 5400): B. breve UBBr-01 (MTCC 25081): B. infantis UBBI-01 (MTCC 25124) was decided to be optimal at 2:2: 2:2: 1:1 in the formulation.

TABLE 1
Individual glutaminase and GABA producing activities, and
synergistic activity of the six strains combined together.

Individual strains

| | GABA | Glutaminase |
|---|---|---|
| Strains | μM/ml | IU/ml |
| Lactobacillus plantarum | nil | 0.023 |
| UBLP-40 | | |
| Lactobacillus rhamnosus | nil | 0.036 |
| UBLR-58 | | |
| B. coagulans Unique IS-2 | 25.30 | 0.051 |
| Bifidobacterium infantis | 33.29 | 0.010 |
| UBBI-01 | | |
| Bifidobacterium breve | 25.85 | 0.021 |
| UBBr-01 | | |
| Bifidobacterium lactis | 17.31 | 0.004 |
| UBBLa-70 | | |
| Combination of 6 strains | 40.45 | 0.056 |

TABLE 2
Combination of two strains and synergistic
activity of the two strain combination

| Combinations of | GABA | Glutaminase |
|---|---|---|
| two strains | μM/ml | IU/ml |
| B. coagulans Unique IS-2 | 29.73 | 0.035 |
| Bifidobacterium. infantis | | |
| UBBI-01 | | |

TABLE 3
Combination of three strains and synergistic
activity of the three strain combination

| Combinations of | GABA | Glutaminase |
|---|---|---|
| three strains | μM/ml | IU/ml |
| Lactobacillus plantarum | 18.78 | 0.023 |
| UBLP-40 | | |
| Lactobacillus rhamnosus | | |
| UBLR-58 | | |
| B. coagulans Unique IS-2 | | |

TABLE 4
Combination of 4 strains, and synergistic
activity of the four strain combination

| | GABA | Glutaminase |
|---|---|---|
| Strains | μM/ml | IU/ml |
| Bacillus coagulans Unique IS-2 | 33.18 | 0.038 |
| Bifidobacterium infantis UBBI-01 | | |
| Bifidobacterium breve UBBr-01 | | |
| Bifidobacterium lactis UBBLa-70 | | |

Thus, it is evident from the experiment and the data given above that different combinations of the strains were tested, and the combination of the six strains showed the maximum synergistic activity for the production of GABA & glutaminase activity. Hence this combination was selected for further studies. Therefore, the particular probiotic consortium disclosed in the current application, at the specific ratios disclosed, is capable of producing GABA at higher levels than the individual strains and also higher than the various other combinations of these strains comprising two, three or four strains.

### Example 2: Study Design

### Materials and Methods

Animals. Healthy pregnant female Sprague-Dawley (SD) rats weighing 200-250 g were used. Both male and female SD rats were used for the study. Pups were born in the animal house facility of NIPER, Hyderabad, India. Except during the period of MS, pups were kept along with their respective mothers until weaning time. Housing rooms were kept at 12/12 h light/dark cycle, with controlled temperature (24±2° C.) and humidity, and rats had free access to food and water ad libitum. The animal experimental design and procedure were approved by the Institutional Animal Ethics Committee (IAEC) of the National Institute of Pharmaceutical Education and Research, Hyderabad, India. All possible efforts were taken to minimize the number of animals and overall suffering to the animals.

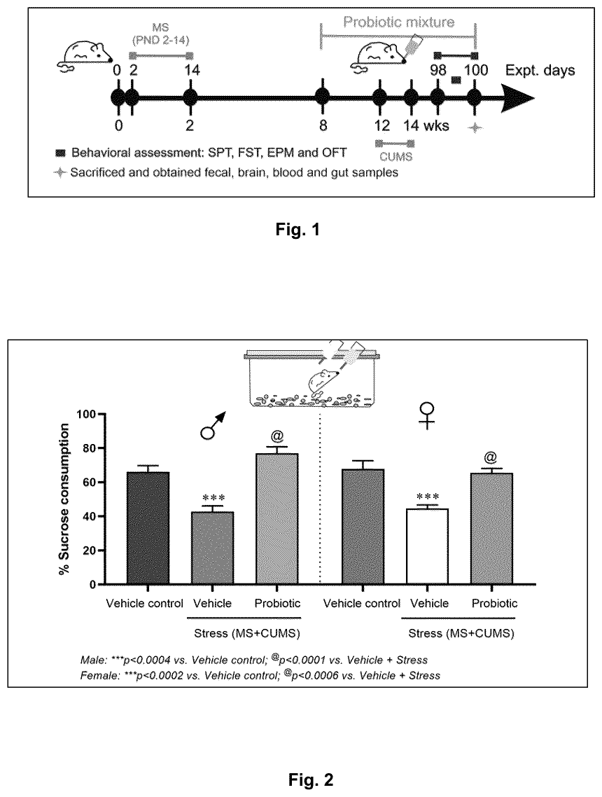

Experimental Design: MS and CUMS. As depicted in FIG. 1, animals were subjected to MS at an early age and then exposed to CUMS at the adult stage to simulate early- and late-life stress. In the first phase of stress exposure (MS), pups were separated from rat dams every day for 3 h between postnatal day 2 (PND2) to PND14. During MS, pups were kept in smaller cages with sterile shredded paper bedding in a different room to avoid ultrasonic vocalization produced by the mother rat. The MS procedure is well accepted and described previously (Ref 5, Dandekar et al). After PND 14, the pups were allowed to stay with their respective mothers until weaning. After weaning, male and female rats were separately group-housed (three to four per cage). Once the animals reached the age of 8 weeks, the probiotic treatment was started as described in the following section. Late-life stress was delivered in the form of CUMS from the 12th week to the 14th week of the study. The CUMS procedure is described Ref 5, Dandekar et al). Briefly, different stressors were delivered in a pseudorandom manner without consecutive repetitions as follows: cold water swimming (4° C. for 5 min), hot water swimming (45° C. for 5 min), tail clamping (1 cm from the tail tip for 2-3 min), foot shock (0.3 mA for 1 min), placing in restrainer at room temperature for 3 h, cage tilting for 12 h, wet bedding for 15 h, cage shaking for 5 min, wet bedding for 12 h, and food restriction for 12 h. Behavioral assessment, that is, FST, SPT, EPM, and OFT, was performed post-CUMS application. At the end of behavioral studies, rats were sacrificed with an overdose of CO2. Then, blood, frontal cortex, hippocampus, fecal samples, and intestine samples were collected and immediately stored at −80° C. until further analysis, and the procedure is already described in Ref 5, Dandekar et al.

Experimental Design: Probiotic Preparation and Treatment. Rats (male and female separately) were randomly divided into three experimental groups (n=6-8 animals per group): [1] vehicle control, [2] vehicle+stress (MS+CUMS), and [3] probiotic+stress (MS+CUMS). For this study, the multi-strain probiotic formulation described in Example 1, was used. This probiotic formulation contained B. coagulans Unique IS-2 (2 billion CFU), L. plantarum UBLP-40 (2 billion CFU), L. rhamnosus UBLR-58 (2 billion CFU), B. lactis UBBLa-70 (2 billion CFU), B. breve UBBr-01 (1 billion CFU), B. infantis UBBI-01 (1 billion CFU), and L-glutamine (250 mg). The probiotic treatment was initiated when animals achieved the age of 7-8 weeks and continued for 6 weeks. The contents of the multi-strain probiotic formulation in the form of lyophilized powder were dissolved in drinking water to deliver one dose to each rat daily. This was prepared fresh every day. The vehicle-treated animals were fed with a carrier matrix. The freshly prepared solution of probiotic and vehicle was provided daily between 4 and 6 pm. The body weights of rats were measured weekly once at 9 AM throughout the study. Statistical Analysis. Data are presented and plotted as mean ±S.E.M. The data were analyzed by one-way ANOVA, followed by post hoc Dunnett's multiple comparisons test. Two-way ANOVA followed by Bonferroni's multiple comparisons test was used to find the sex specific correlation. p<0.05 was considered statistically significant.

### Example 2: Behavioral Assessment

2.1: Sucrose Preference Test (SPT): Anhedonia is one of the core symptoms of depression, which can be evaluated using the SPT in rats. The test was a 3-day procedure. On habituation day (day 1), free access to 1% w/v sucrose solution was given through two drinking bottles containing 200 mL solution. On day 2, the bottle containing 1% w/v sucrose solution (200 mL) and a bottle containing regular drinking water (200 mL) were kept. The position was interchanged every 6 h to negate the effects of side preference. Before test day (i.e., day 3), the rats were deprived of food and water for 12 h. On the test day, the same procedure was adopted as day 2. The pre-weighed bottles were placed, and the intake of sucrose solution, drinking water, and total consumption of liquid were calculated after 12 h again. The % sucrose preference was calculated as follows: % sucrose consumed=[final weight of sucrose (after 12 h)—initial weight of sucrose/weight of sucrose+weight of water]×100.

It was observed that application of MS+CUMS significantly decreased the amount of sucrose solution consumption in both male (p<0.0004) and female (p<0.0002) rats as compared to that of vehicle control animals (FIG. 2). This indicates the development of anhedonic-like behaviors in both sexes. Six weeks of multi-strain probiotic treatment normalized the anhedonia phenotype as indicated by the significant increase in sucrose preference in both male (34%, p<0.0001) and female (21%, p<0.0006) rats versus respective MS+CUMS-exposed rats (FIG. 2). No sex-specific interaction was observed after applying two-way ANOVA, followed by Bonferroni's multiple comparisons test [F (1,51)=1.108, p=0.2975].

2.2 Forced Swimming Test (FST): The FST is the most widely used assay for predicting the antidepressant-like phenotype in rodents. The increased passivity (immobility) in the swimming tank indicates behavioral despair. The method is also described in Ref 5, Dandekar et al: (incorporated herein its entirety), and has also been employed to screen the antidepressant-like effects of prebiotics and probiotics. On day 1 of the test, rats were immersed in a cylindrical tank of dimensions 50 cm in height and 25 cm in diameter containing around 30 cm of water with a temperature of 25° C. and allowed to swim for 15 min. After 24 h, individual rats were re-subjected to the same swimming cylinder for a test period of 5 min, and the immobility time was recorded manually by an observer who was blind to the treatment condition.

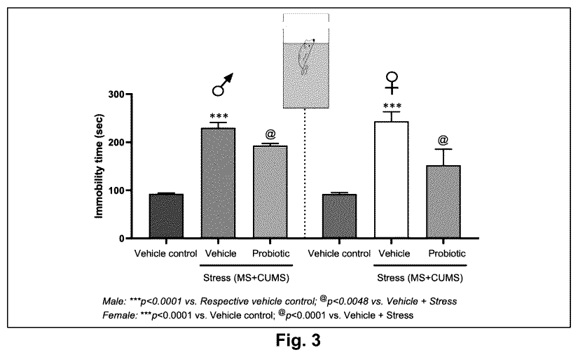

The MS+CUMS-exposed rats showed a significant increase in immobility time in both male (148%, p<0.0001) and female (164%, p<0.0001) rats as compared to the vehicle control group. This indicates the development of behavioral despair condition in rats. Treatment with the multi-strain probiotic formulation significantly reversed the passivity in both male (p<0.0048) and female animals (p<0.0001) as compared to that in the MS +CUMS group (FIG. 3). Two way ANOVA showed no significant effect of the sex factor [F (1,30)=2.049, p=0.1627].

2.3: Elevated Plus Maze (EPM): The EPM is one of the most commonly used rodent tests for assessing anxiety-like behavior and is well described in Ref 5, Dandekar et al. The apparatus consists of two opposing open arms (50 cm×10 cm) and two opposing wall-enclosed arms (50 cm×10 cm×40 cm). The maze is placed on a 50 cm high pedestal. First, animals were acclimatized to the testing room for 30-45 min prior to the experiment. Then, each rat was placed on the center platform facing toward one of the open arms. The number of entries in open and closed arms and the time spent in each arm were recorded by an observer blind to the animal groups for 5 min. After each trial, the apparatus was wiped with 30% isopropanol to avoid the impact of odor.

The EPM test was performed to assess anxiety-like phenotypes in rats. As expected, a significant decrease in the number of entries (p<0.0001 in both male and female rats, Table 2) and time spent in open arms (p<0.0001 in both male and female rats) was observed in MS+CUMS-exposed animals as compared to the vehicle control group. This neurobehavioral aberration in MS+CUMS-exposed animals was significantly ameliorated by the end of treatment in the multistrain probiotic formulation-treated animals, as an increase in the number of entries (p<0.0215 and p<0.0001 in male and female rats, respectively) and the time spent in the open arms [p<0.0001 (93%) and p<0.006 (35%) in male and female rats, respectively] was observed compared to the vehicle +stress (MS+CUMS) group. The number of closed arm entries remained non-significant across the groups (Table 5: each value represented as mean ±S.E.M for six to eight rats. Male: *** p<0.0001 and #p<0.0001 vs vehicle control: (@p<0.0215 and ∧p <0.0001 vs vehicle+stress; Female: *** p<0.0001 and ++p<0.0001 vs vehicle control; @p<0.0001 and ** p<0.006 vs vehicle+stress).

TABLE 5
Effect of Multi-strain Probiotics Treatment on Exploratory
Behavior of Male and Female Rats for 5 min in the EPM

| | | % Entries in open | | % Time spent in | | Total entries in | |
|---|---|---|---|---|---|---|---|
| | | arms (Mean ± | | open arms (Mean ± | | closed arms (Mean ± | |
| | | S.E.M.) | | S.E.M.) | | S.E.M.) | |
| SN | Treatment | Male | Female | Male | Female | Male | Female |
| 1 | Vehicle control | 59.41 ± | 58.88 ± | 51.94 ± | 50.72 ± | 3.16 ± | 3.66 ± |
| | | 1.07 | 2.00 | 1.26 | 1.89 | 0.16 | 0.33 |
| 2 | Vehicle + Stress | 35.26 ± | 14.78 ± | 22.00 ± | 30.15 ± | 5.33 ± | 5.50 ± |
| | (MS + CUMS) | 2.30*** | 1.87*** | 3.62*** | 2.79*** | 0.91 | 0.71 |
| 3 | Probiotics + Stress | 42.67 ± | 36.24 ± | 42.39 ± | 40.72 ± | 5.50 ± | 5.80 ± |
| | (MS + CUMS) | 1.57^{@} | 2.35^{@} | 1.82^{@} | 1.01^{@} | 0.71 | 0.73 |

2.4: Open Field Test (OFT) is used to evaluate ambulatory activity in a perplex box (50×50 cm). Each rat was placed in the center of the box and allowed to explore the arena for 10 min without previous habitation. The arena was wiped and cleaned with 30% v/v isopropanol between each trial to avoid smell cues. The horizontal activity of rats was recorded using a top-mounted video camera connected to an ANY-maze behavioral tracking software (Stoelting Co., United Kingdom). The total distance travelled by each animal was calculated and presented.

In the OFT, we did not observe any significant change in the horizontal activity (p>0.05) of animals as the total distance travelled by rats across the groups was similar (Table 6; the total distance travelled during test time is calculated and presented as mean ±S.E.M. for six to eight rats).

Effect of multi-strain probiotic formulation Administration on Body Weight. The body weight was recorded once weekly. We did not observe a noticeable change in the body weight of rats across the different treatment groups (data not shown).

TABLE 6
Effect of Multi-strain Probiotics Treatment on Ambulatory
Activity of Male and Female Rats for 10 min in the OFT

| | | Distance travelled (cm) | |
|---|---|---|---|
| | | (Mean ± S.E.M.) | |
| SN | Treatment | Male | Female |
| 1 | Vehicle control | 468.6 ± 8.25 | 529.6 ± 17.73 |
| 2 | Vehicle + Stress (MS + CUMS) | 429.9 ± 10.48 | 486.1 ± 22.18 |
| 3 | Probiotics + Stress (MS + CUMS) | 442.4 ± 21.71 | 464.0 ± 10.14 |

### Example 3: Estimation of TNF-α, CRP, and BDNF in the Frontal Cortex and Hippocampus Using ELISA

The hippocampus and frontal cortex brain samples were homogenized using T-PER (Thermo Scientific, 78510) 2 mL per 100 mg of tissue and a 5 μL protease inhibitor cocktail (Sigma, P8340). Samples were centrifuged for 20 min at 13,000 rpm at 4° C. The supernatant solution was obtained and stored at −80° C. until further use. Protein estimation was performed by the Rapid Gold BCA protein assay kit (Thermo Scientific, A53225). Briefly, 100 μL of the sample was added and incubated for 2 h at room temperature. After washing, 100 μL of detection antibody was added. After 2 h of incubation, the plates were washed three times, followed by adding 100 μL of streptavidin-HRP B, keeping for 20 min at room temperature, and protecting from direct light. The assay was terminated by adding 100 μL of substrate solution for 20 min and 50 μL of stop solution to each well. Optical density was measured using a microplate reader at 450 nm.

Cytokine levels were estimated in the frontal cortex and hippocampus as per the given instructions in the commercially available ELISA kits. The Quantikine rat TNF-α R&D Systems (DY510-05) and CRP ELISA kits (DY1744) were used for this estimation. BDNF levels were also estimated in the frontal cortex and hippocampus samples using the R&D systems (DBNT00) BDNF ELISA kit. The results were calculated based on the absorbance of complex antigens and antibodies.

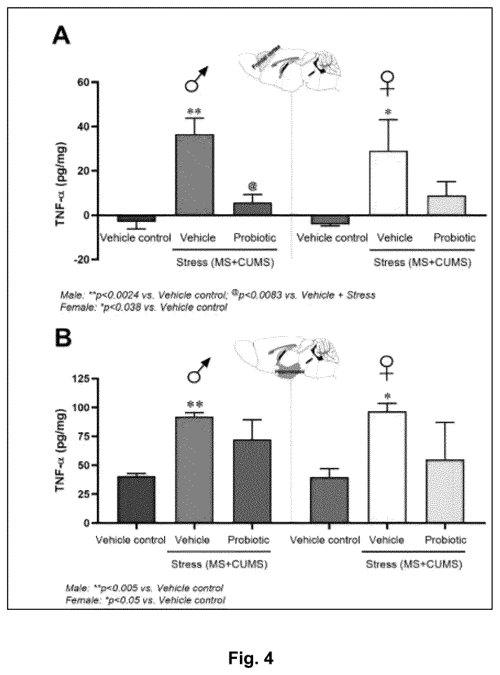

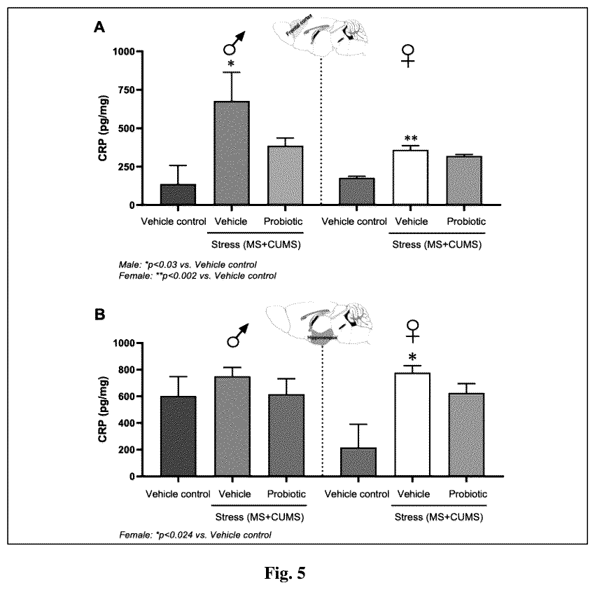

We assessed the levels of CRP and TNF-α cytokines in the frontal cortex and hippocampus of the brain by the ELISA method. A significant increase in the concentration of TNF-α cytokine was observed in the frontal cortex (p<0.0025 and p<0.038 in male and female rats, respectively) and hippocampus samples (p<0.019 and p<0.002 in male and female rats, respectively) of MS+CUMS exposed rats compared to the vehicle control group. Six-week administration of the multi-strain probiotic formulation significantly reversed the elevated levels of TNF-α in the frontal cortex (p<0.013 in male rats) and hippocampus samples (p<0.026 and p<0.016 in males and females, respectively) compared to the vehicle+stress (MS+CUMS) group (FIG. 4A, B). Similar to TNF-α, elevated levels of CRP cytokine were observed in both the frontal cortex (p<0.03 and p<0.002 in male and female rats, respectively) and the hippocampus region (p<0.024 in female rats) of MS+CUMS-exposed rats (FIG. 5A).

We also observed reduced levels of CRP in the frontal cortex and hippocampus regions of multi-strain probiotic formulation-administered animals compared to the vehicle+stress (MS+CUMS) group. We also estimated the concentration of BDNF in the frontal cortex and hippocampus (Table 7). A significant decrease in the levels of BDNF was observed in the hippocampus (p<0.0058 vs vehicle control) of MS+CUMS-exposed male rats. Six week administration of the multi-strain probiotic formulation reversed the decreased levels of BDNF in the frontal cortex. However, we did not observe any noticeable change in the frontal cortex of both male and female rats.

In Table 7, the data were analyzed by one-way ANOVA, followed by post-hoc Dunnett's multiple comparisons test. The sex-specific correlation was assessed using two-way ANOVA, followed by Bonferroni's multiple comparisons test. ** p<0.0058 vs respective vehicle control group).

TABLE 7
Effect of the multi-strain probiotic formulation on the Concentration of BDNF
in the Frontal Cortex (A) and Hippocampus (B) in Male and Female Rats

| | | BDNF (pg/mg) in Frontal | | BDNF (pg/mg) in | |
|---|---|---|---|---|---|
| | | cortex (Mean ± S.E.M.) | | Hippocampus (Mean ± S.E.M.) | |
| S. no. | Treatment | Male | Female | Male | Female |
| 1 | Vehicle control | 271.8 ± | 280.9 ± | 510.1 ± | 399.6 ± |
| | | 9.92 | 37.24 | 41.97 | 74.64 |
| 2 | Vehicle + Stress | 239.0 ± | 248.0 ± | 318.2 ± | 345.2 ± |
| | (MS + CUMS) | 19.55 | 44.10 | 32.52** | 21.45 |
| 3 | Probiotics + Stress | 249.7 ± | 282.7 ± | 439.7 ± | 380.1 ± |
| | (MS + CUMS) | 49.49 | 4.08 | 12.69 | 55.92 |

### Example 4: Estimation of Dopamine (DA), Noradrenaline/Norepinephrine (NE)

and Serotonin (5-HT) Using HPLC-ECD. The monoamine concentration of DA, NE, and 5-HT was estimated in the frontal cortex using HPLC coupled with an electrochemical detector as described in Ref 5, Dandekar et al. Briefly, the frontal cortex samples were homogenized in five volumes of diluents (0.2 M perchloric acid containing 0.2 mg/mL L-cysteine). The homogenates were centrifuged at 14,000 rpm for 15 min at 4° C. The supernatants were transferred to pre-labeled vials and injected into the HPLC-ECD system [BASi Epsilon ECD detector (PK/2008/006)]. The calibration accuracy was 97.67-105% for DA, 97.36-102.72% for NE, and 97.76-105% 5-HT. Data was analyzed using an Agilent EZChrom Elite 3.2.0. The results were expressed as nanograms (ng) of neurotransmitters per gram of tissue.

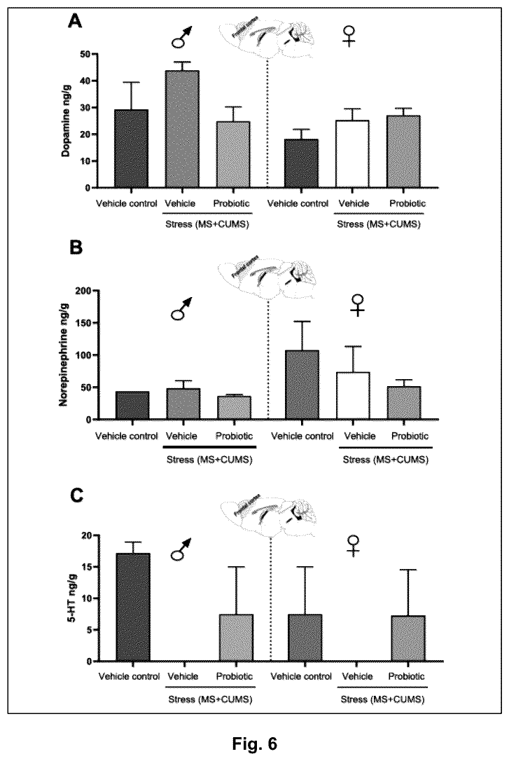

We observed a decrease in the content of 5-HT and increased levels of DA in the frontal cortex of both male and female stress-exposed (MS+CUMS) rats. However, no statistically significant difference was observed due to inter-animal variations. The altered levels of 5-HT and DA in the brain structure were restored to control levels in multi-strain probiotic formulation-treated rats (FIG. 6A, C). However, we did not see any change in the levels of NE in the frontal cortex across the different treatment groups (FIG. 6B).

Elevated systemic inflammatory cytokines disrupt the blood-brain barrier integrity and alter the brain neurotransmitters levels, including 5-HT. Stress-induced depression is a behavioral consequence correlated with the altered levels of monoaminergic neurotransmitters such as 5-HT, NE, and DA (Ref 26 & 27, Dantzer et al & Miller et al).

The production and secretion of these important neurotransmitters are influenced by the gut microbiota (Ref 28, Strandwitz), and importantly, >90% of 5-HT in the human body is produced in the gut. Lowered levels of 5-HT have been documented in depressed patients (Ref 29, Yano et al). In this study, we observed a reduction of 5-HT in the frontal cortex of MS+CUMS-exposed rats, with the levels being restored to normal in multi-strain probiotic formulation-administered animals. The gut microbiota is reported to significantly impact the 5-HT levels by sequestering tryptophan, the precursor of 5-HT. Moreover, increased inflammatory cytokine levels, as can be seen in this study, cause activation of the kynurenine pathway (Ref 28, Strandwitz). Since depression resulted in aberrant tryptophan metabolism encompassing both neuroprotective-like kynurenic acid and neurotoxicity, for example, 3-hydroxy kynurenine metabolites, we estimated the levels of L-tryptophan, L-kynurenine, kynurenic acid, and 3-hydroxy anthranilic acid in the systemic circulation (Ref 30, Quak et al). The levels of these tryptophan metabolites were significantly increased in the stressed animals and restored to normal levels in the multi-strain probiotic formulation-treated animals.

### Example 5: Estimation of L-Tryptophan, L-Kynurenine, Kynurenic Acid, and 3-Hydroxyanthranilic Acid Using LC-MS/MS

Blood plasma was subjected to protein precipitation by adding chilled acetonitrile. In brief, a 50 μL aliquot of plasma sample was extracted with 150 μL of acetonitrile containing 100 ng/ml of tolbutamide as an internal standard (IS), followed by vortex mixing for 10 min. The mixture was centrifuged at 10,000 rpm at 4° C. for 10 min. The resulting supernatant was then transferred to an autosampler vial for LC-MS/MS analysis. Liquid chromatography coupled to mass spectrometry analysis was carried out using the Agilent1200 series (Agilent Technologies, Santa Clara, California, USA), consisting of a quadrupole time-of-flight (Q-TOF) mass spectrometer (6540 series, Agilent Technologies), carried out under the electrospray ionization (ESI) positive mode. Chromatographic separation of L-tryptophan, L kynurenine, kynurenic acid, 3-hydroxyanthranilic acid, and internal standard tolbutamide was achieved on a Waters ACQUITY UPLC BEH C18 column (2.1 mm×100 mm, 1.7 μm). The mobile phase components 0.1% formic acid (A) and acetonitrile (B) were used in the following gradient elution method (Tmin/acetonitrile %): 0.0-0.0/2, 0.0-0.5/2, 0.5-2.0/20, 2.0-2.5/20, 2.5-3.0/35, 3.0-3.5/35, 3.5-4.0/55, 4.0-4.5/55, 4.5-5.0/2, 5.0-6.0/2, 6.0-8.0/98, 8.0-10.0/2, and 10.0-15.0/2. The flow rate was set at 0.2 mL/min, and the column temperature was 30+2° C. The autosampler temperature and injection volume were set as 5+2° C. and 10 μL, respectively. Calibration samples were prepared by spiking 5 μL of working standard solutions of each metabolite (L-tryptophan, L-kynurenine, kynurenic acid, and 3 hydroxyanthranilic acid) into 30 μL of blank rat plasma resulting in the final concentrations of 1, 2.5, 5, 10, 25, 50, 100, 250, 500, 1000, and 2000 ng/mL. The peak area ratios of metabolites to the IS were plotted against theoretical concentrations. The correlation coefficient (R2) was ≥0.99 for all the metabolites.

We estimated the levels of L-tryptophan and its metabolites in blood plasma by the LC-MS/MS method (Table 8). A notable increase in the plasma levels of L tryptophan (p <0.045 in female rats), L-kynurenine (p<0.0001 and p<0.0005 in male and female rats, respectively), kynurenic acid (p<0.0002 in female rats), and 3 hydroxyanthranilic acid (p<0.0001 and p<0.0013 in male and female rats, respectively) was observed in MS+CUMS exposed animals as compared to the vehicle control. However, a significant decrease in the plasma levels of kynurenic acid (p<0.0001) and no alteration in the plasma levels of L tryptophan (p >0.05) were observed in MS+CUMS-exposed male rats. The altered levels of these tryptophan metabolites were restored to near-normal levels by 6 weeks in probiotic treated animals.

In Table 8, the percentage with respect to vehicle control is calculated and presented as mean ±S.E.M for six rats. L-tryptophan (Female: * p<0.045 vs vehicle control), L-kynurenine (male: *** p<0.0001 vs vehicle control: @p<0.0001 vs vehicle+stress; and female: *** p<0.0005 vs vehicle control: (@p<0.0001 vs vehicle+stress), kynurenic acid (male: **** p<0.0001 vs vehicle control; @p<0.0001 vs vehicle+stress; and female: ** p<0.0002 vs vehicle control; @p<0.0001 vs vehicle+stress), and 3-hydroxyanthranilic acid (male: **** p<0.0001 vs vehicle control; @p<0.0001 vs vehicle+stress; and female: ** p<0.0013 vs vehicle control; *** p<0.0007 vs vehicle+stress) in blood plasma of male and female rats. The percentage with respect to vehicle control is calculated and presented as mean ±S.E.M for six rats.

TABLE 8
Effect of Multi-strain Probiotics Treatment on the Concentration of L-
Tryptophan, L-Kynurenine, Kynurenic Acid, and 3-Hydroxyanthranilic Acid

| | | | | | | % Kynurenic | | % 3-Hydroxyanthranilic | |
|---|---|---|---|---|---|---|---|---|---|
| | | % L-Tryptophan | | % L-Kynurenine | | acid | | acid | |
| | | (Mean ± S.E.M.) | | (Mean ± S.E.M.) | | (Mean ± S.E.M.) | | (Mean ± S.E.M.) | |
| S. No | Treatment | Male | Female | Male | Female | Male | Female | Male | Female |
| 1 | Vehicle | 100.00 ± | 100.00 ± | 100.00 ± | 100.00 ± | 100.00 ± | 100.00 ± | 100.00 ± | 100.00 ± |
| | control | 10.80 | 11.48 | 2.53 | 6.92 | 2.26 | 3.54 | 2.65 | 6.94 |
| 2 | Vehicle + | 98.77 ± | 128.30 ± | 152.98 ± | 130.35 ± | 57.27 ± | 119.29 ± | 155.84 ± | 135.56 ± |
| | Stress | 6.38 | 5.62* | 4.96*** | 7.66*** | 1.50**** | 2.24*** | 5.24**** | 7.02** |
| | (MS + CUMS) | | | | | | | | |
| 3 | Probiotics + | 94.24 ± | 104.70 ± | 78.57 ± | 97.31 ± | 83.66 ± | 97.27 ± | 84.15 ± | 97.30 ± |
| | Stress | 4.99 | 5.14 | 4.29^{@} | 1.85^{@} | 2.11^{@} | 1.87^{@} | 1.67^{@} | 1.85*** |
| | (MS + CUMS) | | | | | | | | |

### Example 6: Estimation of the Bacterial Population by Real-Time PCR

DNA was extracted from fecal samples using the QIAamp Power Fecal Pro DNA Kit (51804, Qiagen). The DNA quality and quantity were determined using the NanoDrop 2000c Spectrophotometer (ND-2000C). Real-time qPCR was performed using the QuantStudio 7 Pro Real-Time PCR System (Applied Biosystems). Amplification and detection were carried out using the iTaq Universal SYBR Green Supermix (Biorad). A final volume of 20 μL was used for each reaction with a 0.25 μM concentration of each primer. The details of each primer are given in Table 6. The amplifications were made using initial denaturation at 95° C. for 30 s, cycling for 43 s, denaturation for 5 s at 95° C., annealing at 63° C. for 30 s, and extension at 72° C. for 30 s, followed by 35 cycles of 95° C. for 30 s at 60° C. for 1 min. The bacterial population was calculated relative to 16S ribosomal RNA as per an earlier published study (Ref 31, Yang et al). The percentage abundance was calculated as (each bacterial population in fecal samples)/(each bacterial population in the highest one)×100.

TABLE 9
Primers

| Phylum | Forward | Reverse |
|---|---|---|
| Firmicutes | SEQ ID NO: 1 | SEQ ID NO: 2 |
| Bacteroidetes | SEQ ID NO: 3 | SEQ ID NO: 4 |
| Actinobacteria | SEQ ID NO: 5 | SEQ ID NO: 6 |
| Proteobacteria | SEQ ID NO: 7 | SEQ ID NO: 8 |
| 16S rRNA | SEQ ID NO: 9 | SEQ ID NO: 10 |

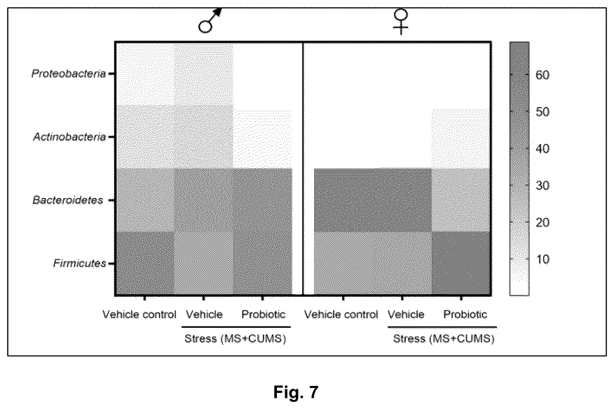

We observed two predominant phyla, that is, Firmicutes and Bacteroidetes, in the fecal samples (FIG. 7). The decreased Firmicutes-to Bacteroidetes ratio was observed in CUMS rats compared to normal animals, while we did not see the change in female rats (Table 10). Administration of the multi-strain probiotic formulation increased the Firmicutes to-Bacteroidetes ratio compared to the CUMS group.

TABLE 10
Firmicutes-to-Bacteroidetes (F/B) Ratio

| | Male | Female |
|---|---|---|
| Treatment | F/B ± S.E.M. | F/B ± S.E.M. |
| Vehicle control | 1.84 ± 0.62 | 0.54 ± 0.05 |
| vehicle + stress | 0.85 ± 0.43 | 0.58 ± 0.13 |
| (MS + CUMS) | | |
| probiotic + stress | 1.04 ± 0.28 | 1.77 ± 2.10 |
| (MS + CUMS) | | |

### Example 7: Quantification of SCFA by Gas Chromatography with Flame Ionization Detection

To investigate the effect of the multi-strain probiotic formulation on bacterial fermentation end products, we quantified the levels of acetate, butyrate, and propionate in rats' fecal samples by using gas chromatography-flame ionization detection (GC-FID). Fecal samples (100 mg) were mixed with 1 mL of water and homogenized for 30 s under 6500 rpm three times and then incubated for 30 min at 4° C. After centrifugation for 30 min at 13,000 g, 500 μL of supernatant (fecal homogenate) was mixed with 5 M HCl to bring the pH of the fecal solution to 2. The acidified fecal homogenate was extracted by adding 1 mL of diethyl ether (DE), vortexed, and incubated on ice for 5 min and then centrifuged for 5 min at 10,000 g. The DE layer (containing SCFAs) was transferred to a new centrifuge tube containing anhydrous Na2SO4. The remaining aqueous layer was further extracted two times using DE. The DE layers were mixed for further derivatization. Then, 1 mL of DE extract was transferred into a glass insert in a GC vial and capped tightly after adding 5 μL of BSTFA (A5603) and vortexed for 5 s. The mixture was kept in the GC vial and incubated at 37° C. for 2 h, and then as described earlier by Zhang et al (Ref 32), the derivatized samples were loaded to GC-FID (8890, Agilent Technologies). The acetate, propionate, and butyrate concentration was determined based on the known amount of SCF As.

As shown in Table 11, lower levels of propionate and butyrate were observed in the CUMS group. Treatment with the multi-strain probiotic formulation significantly increased the levels of acetate (p<0.016) in male rats and butyrate (p<0.04 and p<0.0042) in male and female rats, respectively. Data shown has *p<0.016, ** p<0.0042, and @p<0.04 vs respective vehicle+stress; #p<0.01 vs vehicle control

TABLE 11
Effect of Multi-strain Probiotics formulation treatment on the Levels of SCFAs (Acetate,
Propionate, and Butyrate) in the Fecal Samples of Male and Female Rats

| | | Acetate (Mean ± | | Propionate (Mean ± | | Butyrate (Mean ± | |
|---|---|---|---|---|---|---|---|
| | | S.E.M.) | | S.E.M.) | | S.E.M.) | |
| SN | Treatment | Male | Female | Male | Female | Male | Female |
| 1 | Vehicle control | 29.55 ± | 28.53 ± | 11.32 ± | 10.71 ± | 5.93 ± | 3.31 ± |
| | | 7.26 | 2.70 | 3.39 | 2.15 | 0.88 | 0.69 |
| 2 | Vehicle + Stress | 39.32 ± | 34.60 ± | 9.27 ± | 8.04 ± | 2.10 ± | 2.96 ± |
| | (MS + CUMS) | 1.74 | 6.07 | 1.92 | 1.96 | 0.16^{#} | 0.66 |
| 3 | Probiotics + | 66.02 ± | 14.77 ± | 19.26 ± | 8.55 ± | 5.15 ± | 8.49 ± |
| | Stress | 4.33* | 4.63 | 3.47 | 1.67 | 0.49^{@} | 0.28** |
| | (MS + CUMS) | | | | | | |

### Example 8: Hematoxylin and Eosin Staining of the Intestine

The distal part of the colon was excised and fixed in 4% paraformaldehyde. The tissue was embedded in paraffin, and 5 μm-thick sections were taken using a microtome. Hematoxylin and eosin (H&E) staining was performed to assess the microscopic changes, including villus height/crypt depth ratio and goblet cell count.

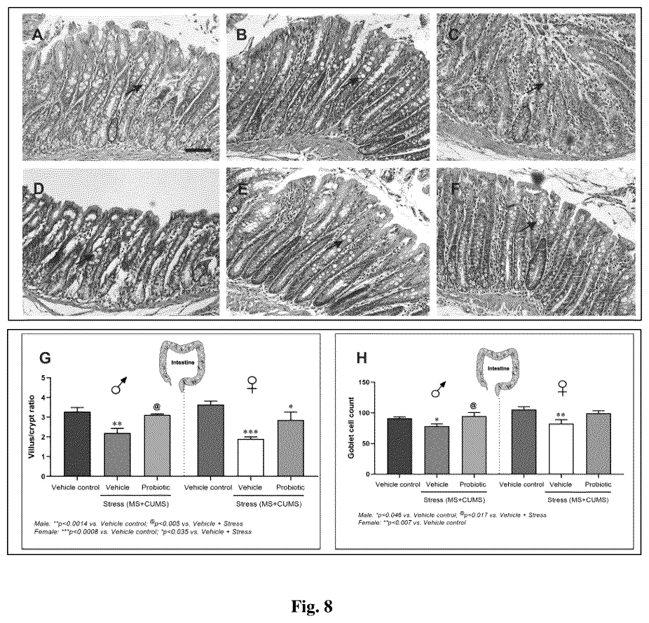

The results of H&E staining in colon tissues are shown in FIG. 8. We observed a significant decrease in the villi/crypt ratio (p<0.0014 and p<0.0008 in male and female rats, respectively) in MS+CUMS exposed animals compared to the vehicle control group (FIG. 8A-H). The villi/crypt ratio appeared normal in multi-strain probiotic-treated male [p<0.005 vs. stress group (MS+CUMS)] and female animals [p<0.035 vs. stress group (MS+CUMS)]. A significant decrease in the number of goblet cells was observed in the vehicle+stress (MS+CUMS) group in both male (p<0.046) and female animals (p<0.007). A comparable goblet cell count was seen in multi-strain probiotic formulation-treated groups (both male and female rats) to vehicle control animals (FIG. 8A-H). This indicates that probiotic treatment may positively help in the renewal of intestinal epithelium and subsequently intestinal integrity.

### REFERENCES

1. Fava, M., Davidson, K. G. (1996) Definition and epidemiology of treatment-resistant depression. Psychiatr Clin North Am. 19 (2): 179-200.
2. Golden, R. N., Gaynes, B. N., Ekstrom, R. D., Hamer, R. M., Jacobsen, F. M., Suppes, T., Wisner, K. L., Nemeroff, C. B. (2005) The efficacy of light therapy in the treatment of mood disorders: a review and meta-analysis of the evidence. Am J Psychiatry. 162 (4): 656-62.
3. Zheng, P., Zeng, B., Zhou, C., Liu, M., Fang, Z., Xu, X., Zeng, L., Chen, J., Fan, S., Du, X., Zhang, X., Yang, D., Yang, Y., Meng, H., Li, W., Melgiri, N. D., Licinio, J., Wei, H., Xie, P. (2016) Gut microbiome remodeling induces depressive-like behaviors through a pathway mediated by the host's metabolism. Mol Psychiatry. 21 (6): 786-96.
4. Jiang, H., Ling, Z., Zhang, Y., Mao, H., Ma, Z., Yin, Y., Wang, W., Tang, W., Tan, Z., Shi, J., Li, L., Ruan, B. (2015) Altered fecal microbiota composition in patients with major depressive disorder. Brain Behav Immun. 48:186-94.
5. Dandekar, M. P., Palepu, M. S. K., Satti, S., Jaiswal, Y., Singh, A. A., Dash, S. P., Gajula, S. N. R., Sonti, R. (2022) Multi-strain Probiotic Formulation Reverses Maternal Separation and Chronic Unpredictable Mild Stress-Generated Anxiety- and Depression-like Phenotypes by Modulating Gut Microbiome-Brain Activity in Rats. ACS Chem Neurosci. 13 (13): 1948-1965.
6. Iordache, M. M., Tocia, C., Aschie, M., Dumitru, A., Manea, M., Cozaru, G. C., Petcu, L., Vlad, S. E., Dumitru, E., Chisoi, A. (2022) Intestinal Permeability and Depression in Patients with Inflammatory Bowel Disease. J Clin Med. 11 (17): 5121
7. Liu, R. T., Rowan-Nash, A. D., Sheehan, A. E., Walsh, R. F. L., Sanzari, C. M., Korry, B. J., Belenky, P. (2020) Reductions in anti-inflammatory gut bacteria are associated with depression in a sample of young adults. Brain Behav Immun. 88:308-324.
8. Chen, L. M., Bao, C. H., Wu, Y. et al. (2021) Tryptophan-kynurenine metabolism: a link between the gut and brain for depression in inflammatory bowel disease. J Neuroinflammation 18:135.
9. Sulthana, A., Lakshmi, S. G., Madempudi, R. S. (2019) High-quality draft genome and characterization of commercially potent probiotic Lactobacillus strains. Genomics Inform. 17 (4): e43.
10. Caldirola, D., De Donatis, D., Alciati, A., Daccò, S., Perna, G. (2023) Pharmacological approaches to the management of panic disorder in older patients: a systematic review. Expert Rev Neurother. 23 (11): 1013-1029.
11. Cryan, J. F., Holmes, A. (2005) The ascent of mouse: advances in modelling human depression and anxiety. Nat Rev Drug Discov. 4 (9): 775-90.
12. Markov, D. D. (2022) Sucrose Preference Test as a Measure of Anhedonic Behavior in a Chronic Unpredictable Mild Stress Model of Depression: Outstanding Issues. Brain Sci. 12 (10): 1287.
13. Vetulani, J. (2013) Early maternal separation: a rodent model of depression and a prevailing human condition. Pharmacol Rep. 65 (6): 1451-61.
14. Willner, P. (2016) The chronic mild stress (CMS) model of depression: History, evaluation and usage. Neurobiol Stress. 6:78-93.
15. Aricioğlu, F., Yalcinkaya, C., Ozkartal, C. S., Tuzun, E., Sirvanci, S., Kucukali, C. I., Utkan, T. (2020) NLRP1-Mediated Antidepressant Effect of Ketamine in Chronic Unpredictable Mild Stress Model in Rats. Psychiatry Investig. 17 (4): 283-291.
16. Huang, J., Shen, C., Ye, R., Shi, Y., Li, W. (2021) The Effect of Early Maternal Separation Combined With Adolescent Chronic Unpredictable Mild Stress on Behavior and Synaptic Plasticity in Adult Female Rats. Front Psychiatry. 12:539299.
17. Bian, Y., Ma, Y., Ma, Q., Yang, L., Zhu, Q., Li, W., Meng, L. (2021) Prolonged Maternal Separation Induces the Depression-Like Behavior Susceptibility to Chronic Unpredictable Mild Stress Exposure in Mice. Biomed Res Int. 2021:6681397.
18. Belovicova, K., Bogi, E., Csatlosova, K., Dubovicky, M. (2017) Animal tests for anxiety-like and depression-like behavior in rats. Interdiscip Toxicol. 10 (1): 40-43.
19. Can, A., Dao, D. T., Arad, M., Terrillion, C. E., Piantadosi, S. C., Gould, T. D. (2012) The mouse forced swim test. J Vis Exp. (59): e3638.
20. Slattery, D. A., Cryan, J. F. (2012) Using the rat forced swim test to assess antidepressant-like activity in rodents. Nat Protoc. 7 (6): 1009-14.
21. Teegarden, S. (2012) Behavioral Phenotyping in Rats and Mice. MATER METHODS, 2:122.
22. Kunugi, H. (2021) Gut Microbiota and Pathophysiology of Depressive Disorder. Ann Nutr Metab. 77 (suppl 2): 11-20.
23. Duval, F., Lebowitz, B. D., Macher, J. P. (2006) Treatments in depression. Dialogues Clin Neurosci. 8 (2): 191-206.
24. Hertz, L. (2013) The Glutamate-Glutamine (GABA) Cycle: Importance of Late Postnatal Development and Potential Reciprocal Interactions between Biosynthesis and Degradation. Front Endocrinol (Lausanne). 4:59.
25. Venkataraman, R., Madempudi, R. S., Neelamraju, J., Ahire, J. J., Vinay, H. R., Lal, A., Thomas, G., Stephen, S. (2021) Effect of Multi-strain Probiotic Formulation on Students Facing Examination Stress: a Double-Blind, Placebo-Controlled Study. Probiotics Antimicrob Proteins. 13 (1): 12-18.
26. Dantzer, R., O'Connor, J. C., Lawson, M. A., Kelley, K. W. (2011) Inflammation-associated depression: from serotonin to kynurenine. Psychoneuroendocrinology. 36 (3): 426-36.
27. Miller, A. H., Maletic, V., Raison, C. L. (2009) Inflammation and its discontents: the role of cytokines in the pathophysiology of major depression. Biol Psychiatry. 65 (9): 732-41.
28. Strandwitz, P. (2018) Neurotransmitter modulation by the gut microbiota. Brain Res. 1693 (Pt B): 128-133.
29. Yano, J. M., Yu, K., Donaldson, G. P., Shastri, G. G., Ann, P., Ma, L., Nagler, C. R., Ismagilov, R. F., Mazmanian, S. K., Hsiao, E. Y. (2015) Indigenous bacteria from the gut microbiota regulate host serotonin biosynthesis. Cell. 161 (2): 264-76.
30. Quak, J., Doornbos, B., Roest, A. M., Duivis, H. E., Vogelzangs, N., Nolen, W. A., Penninx, B. W., Kema, I. P., de Jonge, P. (2014) Does tryptophan degradation along the kynurenine pathway mediate the association between pro-inflammatory immune activity and depressive symptoms? Psychoneuroendocrinology. 45:202-10.
31. Yang, Y. W., Chen, M. K., Yang, B. Y., Huang, X. J., Zhang, X. R., He, L. Q., Zhang, J., Hua, Z. C. (2015) Use of 16S rRNA Gene-Targeted Group-Specific Primers for Real-Time PCR Analysis of Predominant Bacteria in Mouse Feces. Appl Environ Microbiol. 81 (19): 6749-56.
32. Zhang. S., Wang. H., Zhu. M. J. (2019) A sensitive GC/MS detection method for analyzing microbial metabolites short chain fatty acids in fecal and serum samples. Talanta. 196:249-254.

We claim:

1. A method of treating, preventing or ameliorating at least one symptom of treatment-resistant depression (TRD) in a subject, the method comprising the step of administering to the subject a multistrain probiotic formulation comprising the bacterial strains Bacillus coagulans, Lactobacillus rhamnosus, Bifidobacterium lactis, Lactobacillus plantarum, Bifidobacterium breve and Bifidobacterium infantis and 100-500 mg of Glutamine.

2. The method of claim 1, wherein the Bacillus coagulans is the strain Bacillus coagulans Unique IS-2 (MTCC 5260), Lactobacillus rhamnosus is the strain Lactobacillus rhamnosus UBLR-58 (MTCC 5402), Bifidobacterium lactis is the strain Bifidobacterium lactis UBBLa-70 (MTCC 5400), Lactobacillus plantarum is the strain Lactobacillus plantarum UBLP-40 (MTCC 5380), Bifidobacterium breve is the strain Bifidobacterium breve UBBr-01 (MTCC 25081) and Bifidobacterium infantis is the strain Bifidobacterium infantis UBBI-01 (MTCC 25124) in the multistrain probiotic formulation.

3. The method of claim 1, wherein the TRD is induced by chronic stress or early-life stress.

4. The method of claim 1, wherein the multistrain probiotic formulation comprises Bacillus coagulans: Lactobacillus rhamnosus: Bifidobacterium lactis: Lactobacillus plantarum: Bifidobacterium breve and Bifidobacterium infantis in the ratio 2:2:2:2:1:1.

5. The method of claim 1, wherein the bacterial strains are present in the multistrain probiotic formulation at 106-1012 colony forming unit (cfu) per dose.

6. The method of claim 1, wherein 1 to 2 doses of the multistrain probiotic formulation are administered to the subject per day.

7. The method of claim 1, wherein the multistrain probiotic formulation is administered to the subject for at least 6 weeks.

8. The method of claim 1, wherein the multistrain probiotic formulation is administered to the subject for 6-8 weeks.

9. The method of claim 1, wherein it comprises administering an effective dosage regimen of the multistrain probiotic formulation to the subject, wherein the effective dosage regimen comprises administering 1 to 2 doses per day of the multistrain probiotic formulation, for 6-8 weeks.

10. The method of claim 1, wherein administration of the effective dosage regimen of the multistrain probiotic formulation does not affect metabolic activity or locomotor activity in the subject.

11. The method of claim 1, wherein the multistrain probiotic formulation is administered to the patient as an adjunct to standard medical treatment (SMT) for depression.

12. The method of claim 1, wherein the multistrain probiotic formulation is administered orally to the patient.

13. The method of claim 1, wherein the multistrain probiotic formulation is administered to the subject in the form of a food product, a dietary supplement, or a pharmaceutically acceptable composition.

14. The method of claim 1, wherein the multistrain probiotic formulation alleviates treatment-resistant depression symptoms by increasing gut eubiosis in the patient.

15. The method of claim 1, wherein the administration of an effective dosage regimen of the multistrain probiotic formulation to the subject leads to an increase in the number of goblet cells and improved crypt-to-villi ratio in the colon of the subject.

16. The method of claim 1, wherein administration of an effective dosage of the probiotic formulation leads to amelioration of one or more of symptoms associated with TRD such as irritability, mood swings, depressed mood, disturbed sleep, listlessness, cognitive changes, short term memory loss, anxiousness, restlessness, tension, and anhedonia.

17. The method of claim 1, wherein administration of an effective dosage of the multistrain probiotic formulation leads to a decrease of the depression symptom of anhedonia in subjects treated with the formulation.

18. The method of claim 1, wherein the administration of the effective dosage regimen of multistrain probiotic formulation alleviates TRD symptoms by modulating the levels of tryptophan metabolites in the subject.

19. The method of claim 1, wherein the administration of the effective dosage regimen of multistrain probiotic formulation ameliorates TRD symptoms in the subject by increasing the levels of 5-HT in the subject.

20. The method of claim 1, wherein the administration of the effective dosage regimen of multistrain probiotic formulation ameliorates TRD symptoms by restoring the elevated levels of L-tryptophan, L-kynurenine, kynurenic acid, and 3-hydroxyanthranilic acid in the circulatory system of the subject.

21. The method of claim 1, wherein the administration of the effective dosage regimen of multistrain probiotic formulation ameliorates TRD symptoms by reversing the accumulation of neurotoxic metabolites associated with anxiety or depression in the subject.

22. The method of claim 21, wherein the neurotoxic metabolite is 3-hydroxy anthranilic acid.

23. The method of claim 1, wherein the administration of the effective dosage regimen of multistrain probiotic formulation ameliorates TRD symptoms in the subject by reversing the decrease in levels of short-chain fatty acids associated with anxiety or depression in the subject.

24. The method of claim 23, wherein the short-chain fatty acids are acetate, propionate and/or butyrate.

25. The method of claim 1, wherein the administration of an effective dosage regimen of the multistrain probiotic formulation to the subject ameliorates TRD symptoms by decreasing levels of proinflammatory cytokines in the subject.

26. The method of claim 1, wherein administration of an effective dosage regimen of the multistrain probiotic formulation ameliorates TRD symptoms in the subject by reversing the elevated mRNA expression levels of TNF-α, and C-reactive protein associated with anxiety and depression in the subject.

27. The method of claim 26, wherein administration of an effective dosage regimen of the multistrain probiotic formulation ameliorates TRD symptoms in the subject by reversing the elevated mRNA expression levels of CRP, TNF-α, and dopamine levels in the hippocampus and/or frontal cortex of subjects.

28. The method of claim 1, wherein the administration of an effective dosage regimen of the multistrain probiotic formulation to the subject leads to increase in the number of goblet cells and improved crypt-to-villi ratio in the colon of the subject.

29. The method of claim 29, wherein administration of an effective dosage regimen of the multi-strain probiotic formulation leads to a restoration of levels of tight junction (TJ) proteins in the subject.

30. The method of claim 1, wherein administration of an effective dosage regimen of the multistrain probiotic formulation reverses the decrease in BDNF levels in the subject.

31. The method of claim 1, wherein administration of an effective dosage regimen of the probiotic formulation reverses the decrease in Firmicutes to Bacteroidetes ratio in subjects showing symptoms of TRD.


SEQUENCE LISTING (10 sequences)

SEQ ID NO: 1 - DNA, 25 bp, synthetic construct
  misc_feature 1..25: note = Firmicutes Fwd Primer
  source 1..25: mol_type = other DNA
ggagyatgtggtttaattcgaagca

SEQ ID NO: 2 - DNA, 20 bp, synthetic construct
  misc_feature 1..20: note = Firmicutes Rev Primer
  source 1..20: mol_type = other DNA
agctgacgacaaccatgcac

SEQ ID NO: 3 - DNA, 22 bp, synthetic construct
  misc_feature 1..22: note = Bacteroidetes Fwd Primer
  source 1..22: mol_type = other DNA
gtttaattcgatgatacgcgag

SEQ ID NO: 4 - DNA, 19 bp, synthetic construct
  misc_feature 1..19: note = Bacteroidetes Rev primer
  source 1..19: mol_type = other DNA
ttaasccgacacctcacgg

SEQ ID NO: 5 - DNA, 18 bp, synthetic construct
  misc_feature 1..18: note = Actinobacteria Fwd Primer
  source 1..18: mol_type = other DNA
tgtagcggtggaatgcgc

SEQ ID NO: 6 - DNA, 21 bp, synthetic construct
  misc_feature 1..21: note = Actinobacteria Rev primer
  source 1..21: mol_type = other DNA
aattaagccacatgctccgct

SEQ ID NO: 7 - DNA, 21 bp, synthetic construct
  misc_feature 1..21: note = Proteobacteria Fwd Primer
  source 1..21: mol_type = other DNA
gctaacgcattaagtrycccg

SEQ ID NO: 8 - DNA, 18 bp, synthetic construct
  misc_feature 1..18: note = Proteobacteria Rev Primer
  source 1..18: mol_type = other DNA
gccatgcrgcacctgtct

SEQ ID NO: 9 - DNA, 52 bp, synthetic construct
  misc_feature 1..52: note = 16S rRNA Fwd Primer
  source 1..52: mol_type = other DNA
tcgtcggcagcgtcagatgtgtataagagacaggtgccagcmgccgcggtaa

SEQ ID NO: 10 - DNA, 54 bp, synthetic construct
  misc_feature 1..54: note = 16S rRNA Rev Primer
  source 1..54: mol_type = other DNA
gtctcgtgggctcggagatgtgtataagagacagggactachvgggtwtctaat